(19) United States
(12) Patent Application Publication
(10) Pub. No.: US 2021/0012326 A1
(43) Pub. Date: Jan. 14, 2021

(54) SYSTEM, METHOD AND PROGRAM PRODUCT FOR PROCESSING HEALTH INSURANCE CLAIMS AND TARGETED ADVERTISEMENT-BASED HEALTHCARE MANAGEMENT USING CRYPTOCURRENCY

(71) Applicant: Enzo Ashford Maxwell Zelocchi, West Hollywood, CA (US)

(72) Inventor: Enzo Ashford Maxwell Zelocchi, West Hollywood, CA (US)

(21) Appl. No.: 16/914,248

(22) Filed: Jun. 26, 2020

Related U.S. Application Data
(63) Continuation-in-part of application No. 16/509,477, filed on Jul. 11, 2019.

(51) Int. Cl. G06Q 20/36; G06Q 20/06; G06Q 20/38; G06Q 20/40; G16H 10/60; H04L 9/30; H04L 9/32; G06F 16/23

(52) U.S. Cl. CPC G06Q 20/3674; G06Q 20/3678; G06Q 20/0655; G06Q 20/3829; G06Q 20/3827; G06Q 20/401; G16H 50/20; G16H 10/60; G06Q 20/363; H04L 9/30; H04L 9/3242; G06F 16/2379; G06Q 2220/00; G06Q 20/389

(57) ABSTRACT

A system, program code, and method steps including connecting to a network; registering as a member node of the network; determining a hash of a public key broadcasted on the network; based on the determining step, matching the public key with an address of a wallet stored on the member node, the wallet comprises at least one of a mobile phone-based wallet, a desktop-based wallet, a hardware-based wallets, a crystal-based wallets; based on the matching step, decrypting the public key; comparing the decrypted public key to a private key stored in the wallet; and based on the comparing step, sending a predetermined amount of cryptocurrency to a sender of the public key.

### CROSS-REFERENCE TO RELATED APPLICATIONS

The present Utility patent application claims priority benefit of the [U.S. provisional application for patent Ser. No. 16/509,477, entitled “System, Method and Program Product for Processing Health Insurance Claims and Targeted Advertisement-Based Healthcare Management”, and filed on Jul. 11, 2019 under 35 U.S.C. 119(e). The contents of this related provisional application are incorporated herein by reference for all purposes to the extent that such subject matter is not inconsistent herewith or limiting hereof.

### INCORPORATION BY REFERENCE OF SEQUENCE LISTING PROVIDED AS ATEXT FILE

Not applicable.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection by the author thereof. The copyright owner has no objection to the facsimile reproduction by anyone of the patent document or patent disclosure for the purposes of referencing as patent prior art, as it appears in the Patent and Trademark Office, patent file or records, but otherwise reserves all copyright rights whatsoever.

### BACKGROUND OF THE RELEVANT PRIOR ART

One or more embodiments of the invention generally relate to cryptocurrency in healthcare related services. More particularly, certain embodiments of the invention relate to methods for using a cryptocurrency for healthcare related transactions.

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

The rise in cost of modern healthcare has become a financial burden for many in modern society, especially in the United States. Many individuals cannot afford to pay for the care that the individuals and their families need, and medical expenses push many into poverty, in some cases exacerbating their current health and financial situations.

Typically, users may exchange cryptocurrency for various purposes including purchase of goods and/or services from vendors or gift or payment between individuals. In some embodiments, cryptocurrency may be exchanged for real currency, such as purchasing cryptocurrency with real currency and/or redeeming cryptocurrency for real currency. Fund transfers in cryptocurrency systems are completed with minimal processing fees, allowing users to avoid the steep fees charged by banks and financial institutions for wire transfers.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that typically, privatized health insurance companies act as an intermediary between health care providers and their patients. Because health care is so expensive, in many cases it may not be feasible for the average individual to pay out of pocket for their medical bills. Health insurance has become one means for reducing this financial burden on patients, but it may be difficult for an individual to afford health insurance that covers all their health care needs. Additionally, it can be burdensome and inefficient to file health insurance claims, as many insurance providers may require systems for transferring medically related data to and from health care providers.

Cryptocurrencies hold the promise of making it easier to transfer funds directly between two parties, without the need for a trusted third party like a bank or credit card company. The fund transfers are instead secured by different forms of incentive systems, like Proof of Work or Proof of Stake.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates an exemplary schematic diagram of an end-user to system to outcome diagram of a virtual intelligent healthcare-related services system directed to improve the effectiveness of providing healthcare services while reducing costs by implementing machine learning, in accordance with an embodiment of the present invention;

FIG. 2 illustrates an example “cloud”-based system for providing an advertising platform to at least partially generate credits for users who view the advertising, the system utilizing blockchain-based solutions, in accordance with an embodiment of the present invention;

FIG. 3 illustrates an example “cloud”-based system for providing a unified electronic platform for universal healthcare and related services using artificial intelligence (“AI”) to better understand patient health risk factors and to generate personalized digital health plans, in accordance with an embodiment of the present invention;

FIG. 4 illustrates an example “cloud”-based system for providing various competitive advantages between different institutions, including private, public and government-owned and/or controlled entities, in accordance with an embodiment of the present invention;

FIG. 5 illustrates a flowchart of an exemplary method that uses blockchain technologies to track, analyze, store, and expedite personal medical records, history and documentation, in accordance with an embodiment of the present invention;

FIG. 6 illustrates a flowchart of an exemplary method to provide a platform for advertisement (“ad”) where companies can pay to advertise with videos, audio clips, and banners for their products to specific target audiences with a survey at the end of each ad, in accordance with an embodiment of the present invention;

FIG. 7 illustrates a flowchart of an exemplary method to provide virtual personal health assistants (VPHAs), which are health care specific virtual assistants and combine voice automation and artificial intelligence in order to follow voice (or text) commands to provide information and assistance, in accordance with an embodiment of the present invention;

FIG. 8 illustrates a flowchart of an exemplary method to make medical diagnostics more accurate and accessible by using machine learning to predict what will happen within based on symptoms and individual genetics, in accordance with an embodiment of the present invention;

FIG. 9 illustrates a flowchart of an exemplary method to implement app-based solutions to provide for healthcare cost transparency to place the entire healthcare industry in tight cost competition, in accordance with an embodiment of the present invention;

FIG. 10 illustrates electronic communication mediums and/or devices related to increasing competition in the entire healthcare industry and/or for recycling unused medication, in accordance with an embodiment of the present invention;

FIG. 11 illustrates a block diagram depicting an exemplary client/server system which may be used by an exemplary web-enabled/networked cryptocurrency transaction system embodiment of the present invention;

FIG. 12 illustrates a block diagram depicting a conventional client/server communication system, which may be used by an exemplary web-enabled/networked cryptocurrency transaction system embodiment of the present invention;

FIG. 13 illustrates an exemplary virtual currency transaction system through cryptocurrency wallets, in accordance with an embodiment of the present invention;

FIG. 14 illustrates an exemplary blockchain, in accordance with an embodiment of the present invention; and

FIG. 15 illustrates a flowchart of an exemplary method for updating a blockchain, in accordance with an embodiment of the present invention.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

All words of approximation as used in the present disclosure and claims should be construed to mean “approximate,” rather than “perfect,” and may accordingly be employed as a meaningful modifier to any other word, specified parameter, quantity, quality, or concept. Words of approximation, include, yet are not limited to terms such as “substantial”, “nearly”, “almost”, “about”, “generally”, “largely”, “essentially”, “closely approximate”, etc.

As will be established in some detail below, it is well settled law, as early as 1939, that words of approximation are not indefinite in the claims even when such limits are not defined or specified in the specification.

For example, see Ex parte Mallory, 52 USPQ 297, 297 (Pat. Off. Bd. App. 1941) where the court said “The examiner has held that most of the claims are inaccurate because apparently the laminar film will not be entirely eliminated. The claims specify that the film is “substantially” eliminated and for the intended purpose, it is believed that the slight portion of the film which may remain is negligible. We are of the view, therefore, that the claims may be regarded as sufficiently accurate.”

Note that claims need only “reasonably apprise those skilled in the art” as to their scope to satisfy the definiteness requirement. See Energy Absorption Sys., Inc. v. Roadway Safety Servs., Inc., Civ. App. 96-1264, slip op. at 10 (Fed. Cir. Jul. 3, 1997) (unpublished) Hybridtech v. Monoclonal Antibodies, Inc., 802 F.2d 1367, 1385, 231 USPQ 81, 94 (Fed. Cir. 1986), cert. denied, 480 U.S. 947 (1987). In addition, the use of modifiers in the claim, like “generally” and “substantial,” does not by itself render the claims indefinite. See Seattle Box Co. v. Industrial Crating & Packing, Inc., 731 F.2d 818, 828-29, 221 USPQ 568, 575-76 (Fed. Cir. 1984).

Moreover, the ordinary and customary meaning of terms like “substantially” includes “reasonably close to: nearly, almost, about”, connoting a term of approximation. See In re Frye, Appeal No. 2009-006013, 94 USPQ2d 1072, 1077, 2010 WL 889747 (B.P.A.I. 2010) Depending on its usage, the word “substantially” can denote either language of approximation or language of magnitude. Deering Precision Instruments, L.L.C. v. Vector Distribution Sys., Inc., 347 F.3d 1314, 1323 (Fed. Cir. 2003) (recognizing the “dual ordinary meaning of th[e] term [“substantially”] as connoting a term of approximation or a term of magnitude”). Here, when referring to the “substantially halfway” limitation, the Specification uses the word “approximately” as a substitute for the word “substantially” (Fact 4). (Fact 4). The ordinary meaning of “substantially halfway” is thus reasonably close to or nearly at the midpoint between the forwardmost point of the upper or outsole and the rearwardmost point of the upper or outsole.

Similarly, the term ‘substantially’ is well recognized in case law to have the dual ordinary meaning of connoting a term of approximation or a term of magnitude. See Dana Corp. v. American Axle & Manufacturing, Inc., Civ. App. 04-1116, 2004 U.S. App. LEXIS 18265, *13-14 (Fed. Cir. Aug. 27, 2004) (unpublished). The term “substantially” is commonly used by claim drafters to indicate approximation. See Cordis Corp. v. Medtronic AVE Inc., 339 F.3d 1352, 1360 (Fed. Cir. 2003) (“The patents do not set out any numerical standard by which to determine whether the thickness of the wall surface is ‘substantially uniform.’ The term ‘substantially,’ as used in this context, denotes approximation. Thus, the walls must be of largely or approximately uniform thickness.”); see also Deering Precision Instruments, LLC v. Vector Distribution Sys., Inc., 347 F.3d 1314, 1322 (Fed. Cir. 2003); Epcon Gas Sys., Inc. v. Bauer Compressors, Inc., 279 F.3d 1022, 1031 (Fed. Cir. 2002). We find that the term “substantially” was used in just such a manner in the claims of the patents-in-suit: “substantially uniform wall thickness” denotes a wall thickness with approximate uniformity.

It should also be noted that such words of approximation as contemplated in the foregoing clearly limits the scope of claims such as saying ‘generally parallel’ such that the adverb ‘generally’ does not broaden the meaning of parallel. Accordingly, it is well settled that such words of approximation as contemplated in the foregoing (e.g., like the phrase ‘generally parallel’) envisions some amount of deviation from perfection (e.g., not exactly parallel), and that such words of approximation as contemplated in the foregoing are descriptive terms commonly used in patent claims to avoid a strict numerical boundary to the specified parameter. To the extent that the plain language of the claims relying on such words of approximation as contemplated in the foregoing are clear and uncontradicted by anything in the written description herein or the figures thereof, it is improper to rely upon the present written description, the figures, or the prosecution history to add limitations to any of the claim of the present invention with respect to such words of approximation as contemplated in the foregoing. That is, under such circumstances, relying on the written description and prosecution history to reject the ordinary and customary meanings of the words themselves is impermissible. See, for example, Liquid Dynamics Corp. v. Vaughan Co., 355 F.3d 1361, 69 USPQ2d 1595, 1600-01 (Fed. Cir. 2004). The plain language of phrase 2 requires a “substantial helical flow.” The term “substantial” is a meaningful modifier implying “approximate,” rather than “perfect.” In Cordis Corp. v. Medtronic AVE, Inc., 339 F.3d 1352, 1361 (Fed. Cir. 2003), the district court imposed a precise numeric constraint on the term “substantially uniform thickness.” We noted that the proper interpretation of this term was “of largely or approximately uniform thickness” unless something in the prosecution history imposed the “clear and unmistakable disclaimer” needed for narrowing beyond this simple-language interpretation. Id. In Anchor Wall Systems v. Rockwood Retaining Walls, Inc., 340 F.3d 1298, 1311 (Fed. Cir. 2003)” Id. at 1311. Similarly, the plain language of claim 1 requires neither a perfectly helical flow nor a flow that returns precisely to the center after one rotation (a limitation that arises only as a logical consequence of requiring a perfectly helical flow).

The reader should appreciate that case law generally recognizes a dual ordinary meaning of such words of approximation, as contemplated in the foregoing, as connoting a term of approximation or a term of magnitude; e.g., see Deering Precision Instruments, L.L.C. v. Vector Distrib. Sys., Inc., 347 F.3d 1314, 68 USPQ2d 1716, 1721 (Fed. Cir. 2003), cert. denied, 124 S. Ct. 1426 (2004) where the court was asked to construe the meaning of the term “substantially” in a patent claim. Also see Epcon, 279 F.3d at 1031 (“The phrase ‘substantially constant’ denotes language of approximation, while the phrase ‘substantially below’ signifies language of magnitude, i.e., not insubstantial.”). Also, see, e.g., Epcon Gas Sys., Inc. v. Bauer Compressors, Inc., 279 F.3d 1022 (Fed. Cir. 2002) (construing the terms “substantially constant” and “substantially below”); Zodiac Pool Care, Inc. v. Hoffinger Indus., Inc., 206 F.3d 1408 (Fed. Cir. 2000) (construing the term “substantially inward”); York Prods., Inc. v. Cent. Tractor Farm & Family Ctr., 99 F.3d 1568 (Fed. Cir. 1996) (construing the term “substantially the entire height thereof”); Tex. Instruments Inc. v. Cypress Semiconductor Corp., 90 F.3d 1558 (Fed. Cir. 1996) (construing the term “substantially in the common plane”). In conducting their analysis, the court instructed to begin with the ordinary meaning of the claim terms to one of ordinary skill in the art. Prima Tek, 318 F.3d at 1148. Reference to dictionaries and our cases indicates that the term “substantially” has numerous ordinary meanings. As the district court stated, “substantially” can mean “significantly” or “considerably.” The term “substantially” can also mean “largely” or “essentially.” Webster's New 20th Century Dictionary 1817 (1983).

Words of approximation, as contemplated in the foregoing, may also be used in phrases establishing approximate ranges or limits, where the end points are inclusive and approximate, not perfect; e.g., see AK Steel Corp. v. Sollac, 344 F.3d 1234, 68 USPQ2d 1280, 1285 (Fed. Cir. 2003) where it where the court said [W]e conclude that the ordinary meaning of the phrase “up to about 10%” includes the “about 10%” endpoint. As pointed out by AK Steel, when an object of the preposition “up to” is nonnumeric, the most natural meaning is to exclude the object (e.g., painting the wall up to the door). On the other hand, as pointed out by Sollac, when the object is a numerical limit, the normal meaning is to include that upper numerical limit (e.g., counting up to ten, seating capacity for up to seven passengers). Because we have here a numerical limit—“about 10%”—the ordinary meaning is that that endpoint is included.

In the present specification and claims, a goal of employment of such words of approximation, as contemplated in the foregoing, is to avoid a strict numerical boundary to the modified specified parameter, as sanctioned by Pall Corp. v. Micron Separations, Inc., 66 F.3d 1211, 1217, 36 USPQ2d 1225, 1229 (Fed. Cir. 1995) where it states “It is well established that when the term “substantially” serves reasonably to describe the subject matter so that its scope would be understood by persons in the field of the invention, and to distinguish the claimed subject matter from the prior art, it is not indefinite.” Likewise see Verve LLC v. Crane Cams Inc., 311 F.3d 1116, 65 USPQ2d 1051, 1054 (Fed. Cir. 2002). Expressions such as “substantially” are used in patent documents when warranted by the nature of the invention, in order to accommodate the minor variations that may be appropriate to secure the invention. Such usage may well satisfy the charge to “particularly point out and distinctly claim” the invention, 35 U.S.C. § 112, and indeed may be necessary in order to provide the inventor with the benefit of his invention. In Andrew Corp. v. Gabriel Elecs. Inc., 847 F.2d 819, 821-22, 6 USPQ2d 2010, 2013 (Fed. Cir. 1988) the court explained that usages such as “substantially equal” and “closely approximate” may serve to describe the invention with precision appropriate to the technology and without intruding on the prior art. The court again explained in Ecolab Inc. v. Envirochem, Inc., 264 F.3d 1358, 1367, 60 USPQ2d 1173, 1179 (Fed. Cir. 2001) that “like the term ‘about,’ the term ‘substantially’ is a descriptive term commonly used in patent claims to ‘avoid a strict numerical boundary to the specified parameter, see Ecolab Inc. v. Envirochem Inc., 264 F.3d 1358, 60 USPQ2d 1173, 1179 (Fed. Cir. 2001) where the court found that the use of the term “substantially” to modify the term “uniform” does not render this phrase so unclear such that there is no means by which to ascertain the claim scope.

Similarly, other courts have noted that like the term “about,” the term “substantially” is a descriptive term commonly used in patent claims to “avoid a strict numerical boundary to the specified parameter.”; e.g., see Pall Corp. v. Micron Seps., 66 F.3d 1211, 1217, 36 USPQ2d 1225, 1229 (Fed. Cir. 1995); see, e.g., Andrew Corp. v. Gabriel Elecs. Inc., 847 F.2d 819, 821-22, 6 USPQ2d 2010, 2013 (Fed. Cir. 1988) (noting that terms such as “approach each other,” “close to,” “substantially equal,” and “closely approximate” are ubiquitously used in patent claims and that such usages, when serving reasonably to describe the claimed subject matter to those of skill in the field of the invention, and to distinguish the claimed subject matter from the prior art, have been accepted in patent examination and upheld by the courts). In this case, “substantially” avoids the strict 100% nonuniformity boundary.

Indeed, the foregoing sanctioning of such words of approximation, as contemplated in the foregoing, has been established as early as 1939, see Ex parte Mallory, 52 USPQ 297, 297 (Pat. Off. Bd. App. 1941) where, for example, the court said “the claims specify that the film is “substantially” eliminated and for the intended purpose, it is believed that the slight portion of the film which may remain is negligible. We are of the view, therefore, that the claims may be regarded as sufficiently accurate.” Similarly, In re Hutchison, 104 F.2d 829, 42 USPQ 90, 93 (C.C.P.A. 1939) the court said “It is realized that “substantial distance” is a relative and somewhat indefinite term, or phrase, but terms and phrases of this character are not uncommon in patents in cases where, according to the art involved, the meaning can be determined with reasonable clearness.”

Hence, for at least the forgoing reason, Applicant submits that it is improper for any examiner to hold as indefinite any claims of the present patent that employ any words of approximation.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will be described in detail below with reference to embodiments thereof as illustrated in the accompanying drawings.

References to a “device,” an “apparatus,” a “system,” etc., in the preamble of a claim should be construed broadly to mean “any structure meeting the claim terms” exempt for any specific structure(s)/type(s) that has/(have) been explicitly disavowed or excluded or admitted/implied as prior art in the present specification or incapable of enabling an object/aspect/goal of the invention. Furthermore, where the present specification discloses an object, aspect, function, goal, result, or advantage of the invention that a specific prior art structure and/or method step is similarly capable of performing yet in a very different way, the present invention disclosure is intended to and shall also implicitly include and cover additional corresponding alternative embodiments that are otherwise identical to that explicitly disclosed except that they exclude such prior art structure(s)/step(s), and shall accordingly be deemed as providing sufficient disclosure to support a corresponding negative limitation in a claim claiming such alternative embodiment(s), which exclude such very different prior art structure(s)/step(s) way(s).

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicant hereby gives notice that new Claims may be formulated to such features and/or combinations of such features during the prosecution of the present Application or of any further Application derived therefrom.

### Definitions

References to “one embodiment,” “an embodiment,” “example embodiment,” “various embodiments,” “some embodiments,” “embodiments of the invention,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least some embodiments of the invention” include the stated particular feature, structure, or characteristic.

References to “user”, or any similar term, as used herein, may mean a human or non-human user thereof. Moreover, “user”, or any similar term, as used herein, unless expressly stipulated otherwise, is contemplated to mean users at any stage of the usage process, to include, without limitation, direct user(s), intermediate user(s), indirect user(s), and end user(s). The meaning of “user”, or any similar term, as used herein, should not be otherwise inferred or induced by any pattern(s) of description, embodiments, examples, or referenced prior-art that may (or may not) be provided in the present patent.

References to “end user”, or any similar term, as used herein, is generally intended to mean late stage user(s) as opposed to early stage user(s). Hence, it is contemplated that there may be a multiplicity of different types of “end user” near the end stage of the usage process. Where applicable, especially with respect to distribution channels of embodiments of the invention comprising consumed retail products/services thereof (as opposed to sellers/vendors or Original Equipment Manufacturers), examples of an “end user” may include, without limitation, a “consumer”, “buyer”, “customer”, “purchaser”, “shopper”, “enjoyer”, “viewer”, or individual person or non-human thing benefiting in any way, directly or indirectly, from use of. or interaction, with some aspect of the present invention.

In some situations, some embodiments of the present invention may provide beneficial usage to more than one stage or type of usage in the foregoing usage process. In such cases where multiple embodiments targeting various stages of the usage process are described, references to “end user”, or any similar term, as used therein, are generally intended to not include the user that is the furthest removed, in the foregoing usage process, from the final user therein of an embodiment of the present invention.

Where applicable, especially with respect to retail distribution channels of embodiments of the invention, intermediate user(s) may include, without limitation, any individual person or non-human thing benefiting in any way, directly or indirectly, from use of, or interaction with, some aspect of the present invention with respect to selling, vending, Original Equipment Manufacturing, marketing, merchandising, distributing, service providing, and the like thereof.

References to “person”, “individual”, “human”, “a party”, “animal”, “creature”, or any similar term, as used herein, even if the context or particular embodiment implies living user, maker, or participant, it should be understood that such characterizations are sole by way of example, and not limitation, in that it is contemplated that any such usage, making, or participation by a living entity in connection with making, using, and/or participating, in any way, with embodiments of the present invention may be substituted by such similar performed by a suitably configured non-living entity, to include, without limitation, automated machines, robots, humanoids, computational systems, information processing systems, artificially intelligent systems, and the like. It is further contemplated that those skilled in the art will readily recognize the practical situations where such living makers, users, and/or participants with embodiments of the present invention may be in whole, or in part, replaced with such non-living makers, users, and/or participants with embodiments of the present invention.

Likewise, when those skilled in the art identify such practical situations where such living makers, users, and/or participants with embodiments of the present invention may be in whole, or in part, replaced with such non-living makers, it will be readily apparent in light of the teachings of the present invention how to adapt the described embodiments to be suitable for such non-living makers, users, and/or participants with embodiments of the present invention. Thus, the invention is thus to also cover all such modifications, equivalents, and alternatives falling within the spirit and scope of such adaptations and modifications, at least in part, for such non-living entities.

References to “healthcare” or “health-care” imply the maintenance or improvement of health via the prevention, diagnosis, and treatment of disease, illness, injury, and other physical and mental impairments in people. Health care is delivered by health professionals in allied health fields. Physicians and physician associates are a part of these health professionals. Dentistry, midwifery, nursing, medicine, optometry, audiology, pharmacy, psychology, occupational therapy, physical therapy and other health professions are all part of health care. It includes work done in providing primary care, secondary care, and tertiary care, as well as in public health.

Access to health care may vary across countries, communities, and individuals, largely influenced by social and economic conditions as well as health policies. Providing health care services means “the timely use of personal health services to achieve the best possible health outcomes”. [Source: Access to Health Care in America. The National Academies Press, US National Academies of Science, Engineering and Medicine. 1993.] Factors to consider in terms of healthcare access include financial limitations (such as insurance coverage), geographic barriers (such as additional transportation costs, possibility to take paid time off of work to use such services), and personal limitations (lack of ability to communicate with healthcare providers, poor health literacy, low income). [Source: “Healthcare Access in Rural Communities Introduction”. Rural Health Information Hub. 2019. Retrieved 2019 Jun. 14.] Limitations to health care services affects negatively the use of medical services, efficacy of treatments, and overall outcome (well-being, mortality rates).

Health care systems are organizations established to meet the health needs of targeted populations. According to the World Health Organization (WHO), a well-functioning health care system requires a financing mechanism, a well-trained and adequately paid workforce, reliable information on which to base decisions and policies, and well-maintained health facilities to deliver quality medicines and technologies. [Source: “Health Topics: Health Systems”. www.who.int. World Health Organization. Retrieved 2013 Nov. 24.]

An efficient health care system can contribute to a significant part of a country's economy, development and industrialization. Health care is conventionally regarded as an important determinant in promoting the general physical and mental health and well-being of people around the world. An example of this was the worldwide eradication of smallpox in 1980, declared by the WHO as the first disease in human history to be completely eliminated by deliberate health care interventions. [Source: World Health Organization. Anniversary of smallpox eradication. Geneva, 18 Jun. 2010.]

References to “healthcare industry” imply an aggregation and integration of sectors within the economic system that provides goods and services to treat patients with curative, preventive, rehabilitative, and palliative care. It includes the generation and commercialization of goods and services lending themselves to maintaining and re-establishing health. [Source: “10 Jahre Nationale branchenkonferenz Gesundheitswirtschaft—Ausgewählte Ergebnisse p. 4” (PDF). BioCon Valley GmbH. Retrieved 21 Aug. 2015.] The modern healthcare industry is divided into many sectors and depends on the interdisciplinary teams of trained professionals and paraprofessionals to meet health needs of individuals and populations. [Source: “Health Care Initiatives, Employment & Training Administration (ETA)—U.S. Department of Labor”; Doleta.gov. Retrieved Feb. 17, 2015.

References to “health economics” imply a branch of economics concerned with issues related to efficiency, effectiveness, value and behavior in the production and consumption of health and healthcare. In broad terms, health economists study the functioning of healthcare systems and health-affecting behaviors such as smoking.

References to “health maintenance organization (“HMO”)” imply a medical insurance group that provides health services for a fixed annual fee. [Source: “BBC News—G-I—Health Maintenance Organization/HMO”. news.bbc.co.uk. Retrieved 22 Mar. 2018.] It is an organization that provides or arranges managed care for health insurance, self-funded health care benefit plans, individuals, and other entities, acting as a liaison with health care providers (hospitals, doctors, etc.) on a prepaid basis. The Health Maintenance Organization Act of 1973 required employers with 25 or more employees to offer federally certified HMO options if the employer offers traditional healthcare options. [Source: Joseph L. Dorsey, “The Health Maintenance Organization Act of 1973 (P.L. 93-222) and Prepaid Group Practice Plan,” Medical Care, Vol. 13, No. 1, (January, 1975), pp. 1-9] Unlike traditional indemnity insurance, an HMO covers care rendered by those doctors and other professionals who have agreed by contract to treat patients in accordance with the HMO's guidelines and restrictions in exchange for a steady stream of customers. HMOs cover emergency care regardless of the health care provider's contracted status.

References to “medical imaging” imply a technique and process of creating visual representations of the interior of a body for clinical analysis and medical intervention, as well as visual representation of the function of some organs or tissues (physiology). Medical imaging seeks to reveal internal structures hidden by the skin and bones, as well as to diagnose and treat disease. Medical imaging also establishes a database of normal anatomy and physiology to make it possible to identify abnormalities. Although imaging of removed organs and tissues can be performed for medical reasons, such procedures are usually considered part of pathology instead of medical imaging. As a discipline and in its widest sense, it is part of biological imaging and incorporates radiology which uses the imaging technologies of X-ray radiography, magnetic resonance imaging, medical ultrasonography or ultrasound, endoscopy, elastography, tactile imaging, thermography, medical photography and nuclear medicine functional imaging techniques as positron emission tomography (PET) and Single-photon emission computed tomography (SPECT). Measurement and recording techniques which are not primarily designed to produce images, such as electroencephalography (EEG), magnetoencephalography (MEG), electrocardiography (ECG), and others represent other technologies which produce data susceptible to representation as a parameter graph vs. time or maps which contain data about the measurement locations. In a limited comparison, these technologies can be considered as forms of medical imaging in another discipline.

References to “disease” imply particular abnormal condition that negatively affects the structure or function of part or all of an organism, and that is not due to any external injury. [Source: “Disease” at Dorland's Medical Dictionary; White, Tim (19 Dec. 2014), “What is the Difference Between an ‘Injury’ and ‘Disease’ for Commonwealth Injury Claims?”, Tindall Gask Bentley. Archived from the original on 27 Oct. 2017. Retrieved on Nov. 6, 2017.] Diseases are often construed as medical conditions that are associated with specific symptoms and signs. [Source: “Disease” at Dorland's Medical Dictionary] A disease may be caused by external factors such as pathogens or by internal dysfunctions. For example, internal dysfunctions of the immune system can produce a variety of different diseases, including various forms of immunodeficiency, hypersensitivity, allergies and autoimmune disorders. In humans, disease is often used more broadly to refer to any condition that causes pain, dysfunction, distress, social problems, or death to the person afflicted, or similar problems for those in contact with the person. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases can affect people not only physically, but also mentally, as contracting and living with a disease can alter the affected person's perspective on life. Death due to disease is called death by natural causes. There are four main types of disease: infectious diseases, deficiency diseases, hereditary diseases (including both genetic diseases and non-genetic hereditary diseases), and physiological diseases. Diseases can also be classified in other ways, such as communicable versus non-communicable diseases. The deadliest diseases in humans are coronary artery disease (blood flow obstruction), followed by cerebrovascular disease and lower respiratory infections. [“What is the deadliest disease in the world?”. WHO; 16 May 2012; Archived from the original on 17 Dec. 2014; Retrieved on: Dec. 7, 2014.] In developed countries, the diseases that cause the most sickness overall are neuropsychiatric conditions, such as depression and anxiety.

References to “preventative healthcare” imply measures taken for disease prevention. [Source: Hugh R. Leavell and E. Gurney Clark as “the science and art of preventing disease, prolonging life, and promoting physical and mental health and efficiency. Leavell, H. R., & Clark, E. G. (1979). Preventive Medicine for the Doctor in his Community (3rd ed.). Huntington, N.Y.: Robert E. Krieger Publishing Company.] Just as health comprises a variety of physical and mental states, so do disease and disability, which are affected by environmental factors, genetic predisposition, disease agents, and lifestyle choices. Health, disease, and disability are dynamic processes which begin before individuals realize they are affected. Disease prevention relies on anticipatory actions that can be categorized as primal, [source: “New parents” secure a lifelong well-being for their offspring by refusing to be victims of societal stress during its primal period”. Primal Prevention] [source: Primal Health Research Database, on primary, secondary, and tertiary prevention. [Source: Hugh R. Leavell and E. Gurney Clark as “the science and art of preventing disease, prolonging life, and promoting physical and mental health and efficiency. Leavell, H. R., & Clark, E. G. (1979). Preventive Medicine for the Doctor in his Community (3rd ed.). Huntington, N.Y.: Robert E. Krieger Publishing Company.]

References to “artificial intelligence” imply a intelligence demonstrated by machines, in contrast to the natural intelligence displayed by humans. Colloquially, the term “artificial intelligence” is often used to describe machines (or computers) that mimic “cognitive” functions that humans associate with the human mind, such as “learning” and “problem solving”. [Source: Russell, Stuart J.; Norvig, Peter (2009). Artificial Intelligence: A Modern Approach (3rd ed.). Upper Saddle River, N.J.: Prentice Hall.] As machines become increasingly capable, tasks considered to require “intelligence” are often removed from the definition of AI, a phenomenon known as the AI effect. [Source: McCorduck, Pamela (2004), Machines Who Think (2nd ed.), Natick, Mass.: A. K. Peters, Ltd.] A quip in Tesler's Theorem says “AI is whatever hasn't been done yet.” [Source: Maloof, Mark. “Artificial Intelligence: An Introduction, p. 37” (PDF)]. For instance, optical character recognition is frequently excluded from things considered to be AI, having become a routine technology. [Source: Schank, Roger C. (1991). “Where's the AI”. AI magazine. Vol. 12 no. 4. p. 38.] Modern machine capabilities generally classified as AI include successfully understanding human speech, [source: Russell, Stuart J.; Norvig, Peter (2009). Artificial Intelligence: A Modern Approach (3rd ed.). Upper Saddle River, N.J.: Prentice Hall.] competing at the highest level in strategic game systems (such as chess and Go), autonomously operating cars, intelligent routing in content delivery networks, and military simulations. Artificial intelligence can be classified into three different types of systems: analytical, human-inspired, and humanized artificial intelligence. [Source: Kaplan Andreas; Michael Haenlein (2018) Siri, Siri in my Hand, who's the Fairest in the Land? On the Interpretations, Illustrations and Implications of Artificial Intelligence, Business Horizons, 62(1)] Analytical AI has only characteristics consistent with cognitive intelligence; generating cognitive representation of the world and using learning based on past experience to inform future decisions. Human-inspired AI has elements from cognitive and emotional intelligence; understanding human emotions, in addition to cognitive elements, and considering them in their decision making. Humanized AI shows characteristics of all types of competencies (i.e., cognitive, emotional, and social intelligence), is able to be self-conscious and is self-aware in interactions with others.

References to “machine learning” imply the scientific study of algorithms and statistical models that computer systems use in order to perform a specific task effectively without using explicit instructions, relying on patterns and inference instead. It is seen as a subset of artificial intelligence. Machine learning algorithms build a mathematical model based on sample data, known as “training data”, in order to make predictions or decisions without being explicitly programmed to perform the task. [Source: The definition “without being explicitly programmed” is often attributed to Arthur Samuel, who coined the term “machine learning” in 1959, but the phrase is not found verbatim in this publication, and may be a paraphrase that appeared later. Confer “Paraphrasing Arthur Samuel (1959), the question is: How can computers learn to solve problems without being explicitly programmed?” in Koza, John R.; Bennett, Forrest H.; Andre, David; Keane, Martin A. (1996). Automated Design of Both the Topology and Sizing of Analog Electrical Circuits Using Genetic Programming. Artificial Intelligence in Design '96. Springer, Dordrecht. pp. 151-170; Bishop, C. M. (2006), Pattern Recognition and Machine Learning, Springer]. Machine learning algorithms are used in a wide variety of applications, such as email filtering, and computer vision, where it is infeasible to develop an algorithm of specific instructions for performing the task. Machine learning is closely related to computational statistics, which focuses on making predictions using computers. The study of mathematical optimization delivers methods, theory and application domains to the field of machine learning. Data mining is a field of study within machine learning, and focuses on exploratory data analysis through unsupervised learning. [Source: Bishop, C. M. (2006), Pattern Recognition and Machine Learning, Springer (stating that machine learning and pattern recognition “can be viewed as two facets of the same field.)][Source: Friedman, Jerome H. (1998). “Data Mining and Statistics: What's the connection?”; Computing Science and Statistics. 29 (1): 3-9] In its application across business problems, machine learning is also referred to as predictive analytics.

References to “blockchain” imply a growing list of records, called blocks, that are linked using cryptography. [Source: “Blockchains: The great chain of being sure about things”. The Economist. 31 Oct. 2015. Archived from the original on 3 Jul. 2016. Retrieved 18 Jun. 2016. “The technology behind bitcoin lets people who do not know or trust each other build a dependable ledger. This has implications far beyond the crypto currency”; Narayanan, Arvind; Bonneau, Joseph; Felten, Edward; Miller, Andrew; Goldfeder, Steven (2016). Bitcoin and cryptocurrency technologies: a comprehensive introduction. Princeton: Princeton University Press.] Each block contains a cryptographic hash of the previous block, [source: Narayanan, Arvind; Bonneau, Joseph; Felten, Edward; Miller, Andrew; Goldfeder, Steven (2016). Bitcoin and cryptocurrency technologies: a comprehensive introduction. Princeton: Princeton University Press.] a timestamp, and transaction data (generally represented as a Merkle tree). By design, a blockchain is resistant to modification of the data. It is “an open, distributed ledger that can record transactions between two parties efficiently and in a verifiable and permanent way”. [Source: Iansiti, Marco; Lakhani, Karim R. (January 2017). “The Truth About Blockchain”. Harvard Business Review. Harvard University. Archived from the original on 18 Jan. 2017. Retrieved 17 Jan. 2017. “The technology at the heart of bitcoin and other virtual currencies, blockchain is an open, distributed ledger that can record transactions between two parties efficiently and in a verifiable and permanent way.”] For use as a distributed ledger, a blockchain is typically managed by a peer-to-peer network collectively adhering to a protocol for inter-node communication and validating new blocks. Once recorded, the data in any given block cannot be altered retroactively without alteration of all subsequent blocks, which requires consensus of the network majority. Although blockchain records are not unalterable, blockchains may be considered secure by design and exemplify a distributed computing system with high Byzantine fault tolerance. Decentralized consensus has therefore been claimed with a blockchain. [Source: Raval, Siraj (2016). “What Is a Decentralized Application?”. Decentralized Applications: Harnessing Bitcoin's Blockchain Technology. O'Reilly Media, Inc. pp. 1-2. ISBN 978-1-4919-2452-5. OCLC 968277125. Retrieved 6 Nov. 2016—via Google Books.]

References to “Medicare” imply a national health insurance program in the United States, begun in 1966 under the Social Security Administration (SSA) and now administered by the Centers for Medicare and Medicaid Services (CMS). It provides health insurance for Americans aged 65 and older, younger people with some disability status as determined by the Social Security Administration, as well as people with end stage renal disease and amyotrophic lateral sclerosis (ALS or Lou Gehrig's disease). In 2018, Medicare provided health insurance for over 59.9 million individuals—more than 52 million people aged 65 and older and about 8 million younger people. [Source: 2019 Annual Report of the Medicare Trustees (for the year 2018), Apr. 22, 2019]. On average, Medicare covers about half of healthcare expenses of those enrolled. Medicare is funded by a combination of a payroll tax, beneficiary premiums and surtaxes from beneficiaries, co-pays and deductibles, and general U.S. Treasury revenue.

References to “data management platform (“DMP”)” imply a technology platform used for collecting and managing data, mainly for digital marketing purposes. [Source: “What is a Data Management Platform?—What is a DMP?”. lotame.com. 22 May 2018. Retrieved on: Jul. 5, 2018]. It allows to generate audience segments, which are used to target specific users in online advertising campaigns. The DMP may use Big Data and Artificial Intelligence algorithms to process big data sets about users from various sources. DMP is used for organizing and monetizing data in Real-Time Bidding system by licensing it to global selling platforms (DSPs). This technology is constantly being developed by entities such as Nielsen and Oracle.

References to “genomics” imply an interdisciplinary field of biology focusing on the structure, function, evolution, mapping, and editing of genomes. A genome is an organism's complete set of DNA, including all of its genes. In contrast to genetics, which refers to the study of individual genes and their roles in inheritance, genomics aims at the collective characterization and quantification of all of an organism's genes, their interrelations and influence on the organism.] Genes may direct the production of proteins with the assistance of enzymes and messenger molecules. In turn, proteins make up body structures such as organs and tissues as well as control chemical reactions and carry signals between cells. Genomics also involves the sequencing and analysis of genomes through uses of high throughput DNA sequencing and bioinformatics to assemble and analyze the function and structure of entire genomes. [Source: National Human Genome Research Institute (8 Nov. 2010). “A Brief Guide to Genomics”. Genome.gov. Retrieved 2011 Dec. 3; Concepts of genetics (10th ed.). San Francisco: Pearson Education. 2012; Culver K W, Labow M A (8 Nov. 2002). “Genomics”. In Robinson R (ed.). Genetics. Macmillan Science Library. Macmillan Reference USA.] Advances in genomics have triggered a revolution in discovery-based research and systems biology to facilitate understanding of even the most complex biological systems such as the brain. [Source: Kadakkuzha B M, Puthanveettil S V (July 2013). “Genomics and proteomics in solving brain complexity”. Molecular BioSystems. 9 (7): 1807-21] The field also includes studies of intragenomic (within the genome) phenomena such as epistasis (effect of one gene on another), pleiotropy (one gene affecting more than one trait), heterosis (hybrid vigor), and other interactions between loci and alleles within the genome.

References to “medical diagnosis (“Dx” or “Dₛ”)” imply the process of determining which disease or condition explains a person's symptoms and signs. It is most often referred to as diagnosis with the medical context being implicit. The information required for diagnosis is typically collected from a history and physical examination of the person seeking medical care. Often, one or more diagnostic procedures, such as medical tests, are also done during the process. Sometimes posthumous diagnosis is considered a kind of medical diagnosis.

References to “virtual health assistants” imply a virtual and/or online-based messaging service or system directed to provide answers responsive to specific patient and/or customer inquiries. For instance, “disease-specific bots can answer queries regarding disease—for both patients and doctors, as well as other health professionals and patient relatives. A child health bot can answer questions about children's health for their parents, with information about a wide range of symptoms and illnesses fed into it.”

Headings provided herein are for convenience and are not to be taken as limiting the disclosure in any way.

The enumerated listing of items does not imply that any or all of the items are mutually exclusive, unless expressly specified otherwise.

It is understood that the use of specific component, device and/or parameter names are for example only and not meant to imply any limitations on the invention. The invention may thus be implemented with different nomenclature/terminology utilized to describe the mechanisms/units/structures/components/devices/parameters herein, without limitation. Each term utilized herein is to be given its broadest interpretation given the context in which that term is utilized.

### Terminology

The following paragraphs provide definitions and/or context for terms found in this disclosure (including the appended claims):

“Comprising” And “contain” and variations of them—Such terms are open-ended and mean “including but not limited to”. When employed in the appended claims, this term does not foreclose additional structure or steps. Consider a claim that recites: “A memory controller comprising a system cache . . . ” Such a claim does not foreclose the memory controller from including additional components (e.g., a memory channel unit, a switch).

“Configured To.” Various units, circuits, or other components may be described or claimed as “configured to” perform a task or tasks. In such contexts, “configured to” or “operable for” is used to connote structure by indicating that the mechanisms/units/circuits/components include structure (e.g., circuitry and/or mechanisms) that performs the task or tasks during operation. As such, the mechanisms/unit/circuit/component can be said to be configured to (or be operable) for perform(ing) the task even when the specified mechanisms/unit/circuit/component is not currently operational (e.g., is not on). The mechanisms/units/circuits/components used with the “configured to” or “operable for” language include hardware—for example, mechanisms, structures, electronics, circuits, memory storing program instructions executable to implement the operation, etc. Reciting that a mechanism/unit/circuit/component is “configured to” or “operable for” perform(ing) one or more tasks is expressly intended not to invoke 35 U.S.C. .sctn. 112, sixth paragraph, for that mechanism/unit/circuit/component. “Configured to” may also include adapting a manufacturing process to fabricate devices or components that are adapted to implement or perform one or more tasks.

“Based On.” As used herein, this term is used to describe one or more factors that affect a determination. This term does not foreclose additional factors that may affect a determination. That is, a determination may be solely based on those factors or based, at least in part, on those factors. Consider the phrase “determine A based on B.” While B may be a factor that affects the determination of A, such a phrase does not foreclose the determination of A from also being based on C. In other instances, A may be determined based solely on B.

The terms “a”, “an” and “the” mean “one or more”, unless expressly specified otherwise.

All terms of exemplary language (e.g., including, without limitation, “such as”, “like”, “for example”, “for instance”, “similar to”, etc.) are not exclusive of any other, potentially, unrelated, types of examples; thus, implicitly mean “by way of example, and not limitation . . . ”, unless expressly specified otherwise.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” and “consisting of” limits the scope of a claim to the specified elements or method steps, plus those that do not materially affect the basis and novel characteristic(s) of the claimed subject matter (see Norian Corp. v Stryker Corp., 363 F.3d 1321, 1331-32, 70 USPQ2d 1508, Fed. Cir. 2004). Moreover, for any claim of the present invention which claims an embodiment “consisting essentially of” or “consisting of” a certain set of elements of any herein described embodiment it shall be understood as obvious by those skilled in the art that the present invention also covers all possible varying scope variants of any described embodiment(s) that are each exclusively (i.e., “consisting essentially of”) functional subsets or functional combination thereof such that each of these plurality of exclusive varying scope variants each consists essentially of any functional subset(s) and/or functional combination(s) of any set of elements of any described embodiment(s) to the exclusion of any others not set forth therein. That is, it is contemplated that it will be obvious to those skilled how to create a multiplicity of alternate embodiments of the present invention that simply consisting essentially of a certain functional combination of elements of any described embodiment(s) to the exclusion of any others not set forth therein, and the invention thus covers all such exclusive embodiments as if they were each described herein.

With respect to the terms “comprising,” “consisting of,” and “consisting essentially of,” where one of these three terms is used herein, the disclosed and claimed subject matter may include the use of either of the other two terms. Thus in some embodiments not otherwise explicitly recited, any instance of “comprising” may be replaced by “consisting of” or, alternatively, by “consisting essentially of”, and thus, for the purposes of claim support and construction for “consisting of” format claims, such replacements operate to create yet other alternative embodiments “consisting essentially of” only the elements recited in the original “comprising” embodiment to the exclusion of all other elements.

Moreover, any claim limitation phrased in functional limitation terms covered by 35 USC § 112(6) (post AIA 112(f)) which has a preamble invoking the closed terms “consisting of,” or “consisting essentially of,” should be understood to mean that the corresponding structure(s) disclosed herein define the exact metes and bounds of what the so claimed invention embodiment(s) consists of, or consisting essentially of, to the exclusion of any other elements which do not materially affect the intended purpose of the so claimed embodiment(s).

Devices or system modules that are in at least general communication with each other need not be in continuous communication with each other, unless expressly specified otherwise. In addition, devices or system modules that are in at least general communication with each other may communicate directly or indirectly through one or more intermediaries. Moreover, it is understood that any system components described or named in any embodiment or claimed herein may be grouped or sub-grouped (and accordingly implicitly renamed) in any combination or sub-combination as those skilled in the art can imagine as suitable for the particular application, and still be within the scope and spirit of the claimed embodiments of the present invention. For an example of what this means, if the invention was a controller of a motor and a valve and the embodiments and claims articulated those components as being separately grouped and connected, applying the foregoing would mean that such an invention and claims would also implicitly cover the valve being grouped inside the motor and the controller being a remote controller with no direct physical connection to the motor or internalized valve, as such the claimed invention is contemplated to cover all ways of grouping and/or adding of intermediate components or systems that still substantially achieve the intended result of the invention.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

Those of skill in the art will appreciate that where appropriate, some embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Where appropriate, embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

“Software” may refer to prescribed rules to operate a computer. Examples of software may include: code segments in one or more computer-readable languages; graphical and or/textual instructions; applets; pre-compiled code; interpreted code; compiled code; and computer programs.

While embodiments herein may be discussed in terms of a processor having a certain number of bit instructions/data, those skilled in the art will know others that may be suitable such as 16 bits, 32 bits, 64 bits, 128s or 256-bit processors or processing, which can usually alternatively be used. Where a specified logical sense is used, the opposite logical sense is also intended to be encompassed.

The example embodiments described herein can be implemented in an operating environment comprising computer-executable instructions (e.g., software) installed on a computer, in hardware, or in a combination of software and hardware. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software program code for carrying out operations for aspects of the present invention can be written in any combination of one or more suitable programming languages, including an object oriented programming languages and/or conventional procedural programming languages, and/or programming languages such as, for example, Hypertext Markup Language (HTML), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Java™, Jini™, C, C++, Smalltalk, Perl, UNIX Shell, Visual Basic or Visual Basic Script, Virtual Reality Markup Language (VRML), ColdFusion™ or other compilers, assemblers, interpreters or other computer languages or platforms.

Computer program code for carrying out operations for aspects of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

A network is a collection of links and nodes (e.g., multiple computers and/or other devices connected together) arranged so that information may be passed from one part of the network to another over multiple links and through various nodes. Examples of networks include the Internet, the public switched telephone network, the global Telex network, computer networks (e.g., an intranet, an extranet, a local-area network, or a wide-area network), wired networks, and wireless networks.

The Internet is a worldwide network of computers and computer networks arranged to allow the easy and robust exchange of information between computer users. Hundreds of millions of people around the world have access to computers connected to the Internet via Internet Service Providers (ISPs). Content providers (e.g., web site owners or operators) place multimedia information (e.g., text, graphics, audio, video, animation, and other forms of data) at specific locations on the Internet referred to as webpages. Websites comprise a collection of connected, or otherwise related, webpages. The combination of all the websites and their corresponding webpages on the Internet is generally known as the World Wide Web (WWW) or simply the Web.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The flowchart and block diagrams in the figures illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

It will be readily apparent that the various methods and algorithms described herein may be implemented by, e.g., appropriately programmed general purpose computers and computing devices. Typically, a processor (e.g., a microprocessor) will receive instructions from a memory or like device, and execute those instructions, thereby performing a process defined by those instructions. Further, programs that implement such methods and algorithms may be stored and transmitted using a variety of known media.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

The functionality and/or the features of a device may be alternatively embodied by one or more other devices which are not explicitly described as having such functionality/features. Thus, other embodiments of the present invention need not include the device itself.

The term “computer-readable medium” as used herein refers to any medium that participates in providing data (e.g., instructions) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random-access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, removable media, flash memory, a “memory stick”, any other memory chip or cartridge, a carrier wave as described hereinafter, or any other medium from which a computer can read.

Various forms of computer readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols, such as Bluetooth, TDMA, CDMA, 3G.

Where databases are described, it will be understood by one of ordinary skill in the art that (i) alternative database structures to those described may be readily employed, (ii) other memory structures besides databases may be readily employed. Any schematic illustrations and accompanying descriptions of any sample databases presented herein are exemplary arrangements for stored representations of information. Any number of other arrangements may be employed besides those suggested by the tables shown. Similarly, any illustrated entries of the databases represent exemplary information only; those skilled in the art will understand that the number and content of the entries can be different from those illustrated herein. Further, despite any depiction of the databases as tables, an object-based model could be used to store and manipulate the data types of the present invention and likewise, object methods or behaviors can be used to implement the processes of the present invention.

A “computer system” may refer to a system having one or more computers, where each computer may include a computer-readable medium embodying software to operate the computer or one or more of its components. Examples of a computer system may include: a distributed computer system for processing information via computer systems linked by a network; two or more computer systems connected together via a network for transmitting and/or receiving information between the computer systems; a computer system including two or more processors within a single computer; and one or more apparatuses and/or one or more systems that may accept data, may process data in accordance with one or more stored software programs, may generate results, and typically may include input, output, storage, arithmetic, logic, and control units.

A “network” may refer to a number of computers and associated devices that may be connected by communication facilities. A network may involve permanent connections such as cables or temporary connections such as those made through telephone or other communication links. A network may further include hard-wired connections (e.g., coaxial cable, twisted pair, optical fiber, waveguides, etc.) and/or wireless connections (e.g., radio frequency waveforms, free-space optical waveforms, acoustic waveforms, etc.). Examples of a network may include: an internet, such as the Internet; an intranet; a local area network (LAN); a wide area network (WAN); and a combination of networks, such as an internet and an intranet.

As used herein, the “client-side” application should be broadly construed to refer to an application, a page associated with that application, or some other resource or function invoked by a client-side request to the application. A “browser” as used herein is not intended to refer to any specific browser (e.g., Internet Explorer, Safari, FireFox, or the like), but should be broadly construed to refer to any client-side rendering engine that can access and display Internet-accessible resources. A “rich” client typically refers to a non-HTTP based client-side application, such as an SSH or CFIS client. Further, while typically the client-server interactions occur using HTTP, this is not a limitation either. The client server interaction may be formatted to conform to the Simple Object Access Protocol (SOAP) and travel over HTTP (over the public Internet), FTP, or any other reliable transport mechanism (such as IBM® MQSeries® technologies and CORBA, for transport over an enterprise intranet) may be used. Any application or functionality described herein may be implemented as native code, by providing hooks into another application, by facilitating use of the mechanism as a plug-in, by linking to the mechanism, and the like.

Exemplary networks may operate with any of a number of protocols, such as Internet protocol (IP), asynchronous transfer mode (ATM), and/or synchronous optical network (SONET), user datagram protocol (UDP), IEEE 802.x, etc.

Embodiments of the present invention may include apparatuses for performing the operations disclosed herein. An apparatus may be specially constructed for the desired purposes, or it may comprise a general-purpose device selectively activated or reconfigured by a program stored in the device.

Embodiments of the invention may also be implemented in one or a combination of hardware, firmware, and software. They may be implemented as instructions stored on a machine-readable medium, which may be read and executed by a computing platform to perform the operations described herein.

More specifically, as will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

In the following description and claims, the terms “computer program medium” and “computer readable medium” may be used to generally refer to media such as, but not limited to, removable storage drives, a hard disk installed in hard disk drive, and the like. These computer program products may provide software to a computer system. Embodiments of the invention may be directed to such computer program products.

An algorithm is here, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like. It should be understood, however, that all of these and similar terms are to be associated with the appropriate physical quantities and are merely convenient labels applied to these quantities.

Unless specifically stated otherwise, and as may be apparent from the following description and claims, it should be appreciated that throughout the specification descriptions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

Additionally, the phrase “configured to” or “operable for” can include generic structure (e.g., generic circuitry) that is manipulated by software and/or firmware (e.g., an FPGA or a general-purpose processor executing software) to operate in a manner that is capable of performing the task(s) at issue. “Configured to” may also include adapting a manufacturing process (e.g., a semiconductor fabrication facility) to fabricate devices (e.g., integrated circuits) that are adapted to implement or perform one or more tasks.

In a similar manner, the term “processor” may refer to any device or portion of a device that processes electronic data from registers and/or memory to transform that electronic data into other electronic data that may be stored in registers and/or memory. A “computing platform” may comprise one or more processors.

Embodiments within the scope of the present disclosure may also include tangible and/or non-transitory computer-readable storage media for carrying or having computer-executable instructions or data structures stored thereon. Such non-transitory computer-readable storage media can be any available media that can be accessed by a general purpose or special purpose computer, including the functional design of any special purpose processor as discussed above. By way of example, and not limitation, such non-transitory computer-readable media can include RAM, ROM, EEPROM, CD-ROM or other optical disk storage, magnetic disk storage or other magnetic storage devices, or any other medium which can be used to carry or store desired program code means in the form of computer-executable instructions, data structures, or processor chip design. When information is transferred or provided over a network or another communications connection (either hardwired, wireless, or combination thereof) to a computer, the computer properly views the connection as a computer-readable medium. Thus, any such connection is properly termed a computer-readable medium.

Combinations of the above should also be included within the scope of the computer-readable media.

While a non-transitory computer readable medium includes, but is not limited to, a hard drive, compact disc, flash memory, volatile memory, random access memory, magnetic memory, optical memory, semiconductor based memory, phase change memory, optical memory, periodically refreshed memory, and the like; the non-transitory computer readable medium, however, does not include a pure transitory signal per se; i.e., where the medium itself is transitory.

### Introduction

Challenges and inefficiencies characteristic of traditional HMOs may include labyrinthine billing, insurance, and new patient intake procedures. When coupled with exceedingly complex therapies and treatment requirements, such HMOs may be difficult for customers and/or patients to participate in, leaving such persons often seeking realistic and reliable cost-effective alternatives. Further, such HMOs may not uniformly implement or even properly consider advanced computer-science based data analytic techniques, inclusive of artificial intelligence (“AI”), machine learning, neural networks, image recognition, blockchain-based technologies, AI-based virtual health assistants, data management platforms (DMPs), genomics, and/or the like for the purposes of streamlining existing processes to achieve cost efficiencies.

Filling that gap is “A-Medicare”, a digital platform described by the presently disclosed embodiments that provides for universal healthcare, referring to a system responsible for creating affordable healthcare for all interested participants. Functional integration and/or usage of advanced computer science-based technologies inclusive of machine learning, AI, and blockchain-based technologies, among others, via the freely distributed and accessible platform of “A-Medicare” allow participants to view and respond to private-party sourced advertising to gain credits to at least partially pay for some, and up to all, of their respective healthcare expenses. “A-Medicare” seeks to provide a comprehensive single source destination regarding all healthcare related needs for participants within the United States (“US”) and may also be reconfigured as necessary for implementation in foreign countries, territories and/or jurisdictions.

“A-Medicare” seeks to implement advanced technology-based solutions to decrease the overall cost of health care and to improve efficiencies in all aspects of procuring, receiving, and paying for healthcare-related services using, for example (but not limitation thereto): machine learning, AI, and blockchain-based technologies. “A-Medicare” will create a universal health care system that results in affordable low-cost healthcare and also provides advertisement (“ad”)-based programs for participants to assist in paying for their healthcare bills, e.g., upon viewing the ads. Efficiencies created upon the usage and/or integration of existing healthcare payment plans, platforms, and/or solutions may result in health insurance companies to continue to make profit without requiring state, federal and/or local governments to raise taxes to fund such programs. Moreover, due to efficiencies created by implementing technology, “A-Medicare” may lower the cost of health care for both participants as well as (but not limitation thereto), the federal government. Such a universal health care solution may allow for the spending of money for multiple healthcare platforms as “A-Medicare” seeks to integrate and/or provide all relevant functionality in a single database, network, application (“app”), software package and/or other digital-medium based and computer-implemented solution, e.g., as software-as-a service (“SaaS”).

Continual longstanding increases in the cost of health care has left many Americans struggling to pay for their medical bills. Many people are struggling to pay their medical bills and have accumulated Moreover, studies have indicated that health care costs are considerably (e.g., potentially by an order of magnitude or more) higher in the United States than in any other developed nation. Unsurprisingly, the vast majority of Americans who are not independently wealthy and/or comprehensively insured greatly need an efficient and affordable medical system, and such facts further support that health care in US has been in need of a universal, disruptive shift for the better.

Applications and/or usages of machine learning based solutions in a fully-integrated data source provides an excellent step towards the success of “A-Medicare”, which is also at least partially based on blockchain technologies. By seeking to provide and maintain a unified healthcare system, “A-Medicare” provides a better way for participants to access valid data to assist in more efficient healthcare service provision. Such efficiencies are largely made possible when computer science, medical research, big data analytics, and AI are merged in a streamlined manner to enhance positivity in patients' healthcare-related outcomes.

Therefore, through technology-based improvements in the effectiveness of procuring, providing, and paying for healthcare services, “A-Medicare” also reduces costs faced by participants and patients.

“A-Medicare”, by undertaking, disseminating, and/or implementing any one or more of the discussed technologies, solutions, systems, and/or methods of use thereof aims to create a unified platform to offer a universal healthcare and related services, which also allows for patient cost savings, to add and achieve convenience and ease in the healthcare industry. Patients may further at least partially pay for healthcare related expenses by viewing private-party (e.g., pharmaceutical company) ads on portable electronic devices, e.g., smartphones. Thus, “A-Medicare” seeks to integrate machine learning technology, AI, and blockchain technology, as well as ad viewership, to reduce the cost of healthcare-related services for, for example (but not limitation thereto) both the federal government and plan participants. Further, in certain configurations, “A-Medicare” may be provided as comprehensive guaranteed healthcare coverage similar to Medicare for all eligible persons in the US.

“A-Medicare's” primary goals include providing universal health care system that is affordable and allows US citizens, US residents, other countries and/or program participants and/or patients (collectively referred to as “plan users”) to handle their respective healthcare bills promptly and conveniently on earth and known universe in space. Plan users may have access to different healthcare platforms from a single database allowing for the sharing of health information and records with doctors and other hospitals worldwide. Plan users may also book doctors' appointments, switch health care policies, recycle unused medication, get best pricing for doctors/medications/hospitals, etc. through “A-Medicare”.

### General Description of the Embodiments

“A-Medicare” seeks to be the ultimate destination regarding healthcare related needs for eligible participants in the US, e.g., citizens, permanent residents, lawfully residing aliens, retirees, students, and so on and so forth. Moreover, “A-Medicare” may be reconfigured as necessary for foreign implementations as well, providing all eligible participants electronic access to any relevant panel and/or portal within “A-Medicare” to manage their healthcare needs and also to be paid for viewing private-party originated ad content for assistance in paying for any health insurance premiums.

Generally, “A-Medicare” offers an Internet-accessible web site/portal/platform for providing unique “A-Medicare” related functionality in a strategic panel system for every participant. Through “A-Medicare”, participants are eligible to manage doctor's appointments for themselves and/or loved ones (e.g., upon having suitable authorization to do so), have their entire medical record available, and, for example (but not limitation thereto), choose the premium and lower-cost alternative options regarding at least: health care insurance, hospital services/surgeries/ER visits, medications, doctor specialist, and pharmacy-related items including drugs. “A-Medicare” incorporate various aspects of at least the following technologies: AI; machine learning; an ad system where private parties can pay to advertise their products and services to specific target audiences; virtual personal health assistants; AI-enabled genetic and/or genome-based medical diagnostic capabilities; personalized digital health plans, based on genetic testing, updated directly to a medical chart and reviewed by a given patient's corresponding primary care doctor; and, blockchain technology, integrated and/or offered in a holistic digital platform to offer a comprehensive long-term healthcare solution.

More particularly, by way of example and not limitation, “A-Medicare” at least partially relies upon a blockchain solution that tracks, analyzes, stores, expedites personal medical records, history and documentation upon qualified user, e.g., an eligible participant such as a US citizen, permanent resident, or other lawful resident and/or private party participant, other countries, on earth and known universe in space etc., interaction and/or automatically, while concurrently providing for users to access private-party originated and paid—for ad content to be financially compensated accordingly, e.g., receiving and/or having credited at least partial payment for healthcare-related services received. “A-Medicare” users may seek, schedule, re-schedule, cancel and/or otherwise alter appointments with medical practitioners through blockchain-based solutions, thus streamlining the flow of medical diagnostics resulting in enhanced overall efficiency and pronounced cost savings.

Moreover, “A-Medicare” provides checks and balances to prevent against and counteract the potential prescription of improper medical exams, unnecessary tests, over prescribing medication and also reward users with financially-based (e.g., money-equivalent) credit towards user healthcare bills and premiums for living a healthy and active lifestyle. In one or more embodiments, medical records of patients and organ-donor information may be stored via blockchain-based technologies, which via the “A-Medicare” digital system and/or platform, allow for immediate donor-to-recipient matching. Accordingly, the decision to where an organ will be donated is immediate such that Organ Procurement Organizations (“OPOs”), and all other entities are notified immediately and automatically. This capability to match donors with recipients will limit the possibility of previously donated organs becoming compromised due to delay in transplantation, e.g., referred to as “wasted organs”, and will create an optimized matching system. “A-Medicare” organ transplantation specific functionality may also facilitate tracking of organ pick up, transportation, conservation status and delivery to a final destination, e.g., a transplant hospital. “A-Medicare” permits for the tracking and processing of each and every health insurance claim made by a user, doctor, healthcare service provider, hospital, medical services provider and/or facility in the US and, in certain embodiments, worldwide. Further, in one or more embodiments, “A-Medicare” allows users to purchase medication for an optimal price throughout the US (e.g., for US-specific configurations) or in other specific countries, etc., and also offers organized tracked shipment using blockchain. Configurations of “A-Medicare” also include a global universal database for users allowing for consultations with doctors worldwide using blockchain. Provisions for the recycling of unused medication using blockchain are also available through A-Medicare. Moreover, in some embodiments, “A-Medicare” may also include the ability to process and expedite, typically with absolute transparency, health insurance claims made by a citizen into any doctor's office, specialist, hospital or medical facility (public or private) nationwide in the USA as well as other nations worldwide.

In one or more embodiments, “A-Medicare” also provides a digital platform for ads where companies pay to advertise with videos, audio clips and banners for their respective products to specific target audiences, where the ads each include a survey that may be completed at the end of each of the ads. Users, upon successfully watching the ad and completing the related survey can earn credit, equivalent to monetary value, that may be usable in “A-Medicare” to, for example (but not limitation thereto) pay for their respective health insurances premiums directly to the health insurance companies and/or “A-Medicare” itself for further distribution to intended recipients.

By way of example and not limitation, “A-Medicare” also provides for complete functional integration with virtual personal health assistants (“VPHAs”), which are health care specific chat-bot type virtual assistants that combine voice automation and AI to follow voice (or text) commands to provide responsive information and assistance. VPHAs, as offered through “A-Medicare”, provide unlimited (e.g., 24/7) access to each user and allow for direct connection with a primary care doctor, care team and 911 emergency responders. Also, VPHAs have the ability to provide reminders to adhere to prescribed treatment plans and/or exercise regimens. In one or more embodiments, the VPHAs may provide reminders to take medications, supplements, and make appointments for check-ups. VPHAs, as offered and maintained through the “A-Medicare” system and/or digital platform, allow for immediate responses to questions, anytime/anywhere. At an organizational level, checks and balances are provided including and not limited to screening the VPHA to user to user, user to provider and provider to provider by screening all participants.

By way of example and not limitation, “A-Medicare” integrates AI, machine learning, genomics and medical diagnostics to provide comprehensive patient detection, prescription, and therapy options and/or care. “A-Medicare” may rely on AI, machine learning, and/or the like to make medical diagnostics more accurate and accessible to predict what may happen within based on detectable symptoms and the individual genetic makeup of a user and/or participant, etc.

“A-Medicare” receives basic medical data, genetic testing and machine learning to predict months, and years out, an individual's predisposition to certain cancers, metabolic processes and chronic diseases. Patients, users, participants and/or the like may thus arrange for immediate visits to physician specialists in particular enumerated areas as heart health, cancer, and general wellness based on the severity of the illness and/or condition, etc. In addition, the ability to create personalized health plans, supplement regimens, and fitness plans may be based at least partially on that detected by “A-Medicare” regarding a patient and/or user's detected genetic makeup. “A-Medicare” may further diagnose and alert medical professionals when a medical emergency is occurring based on data input by the patient and/or user and/or another party into an appropriately configured device running “A-Medicare” related software, e.g., an app.

In one or more embodiments, “A-Medicare” offers a streamlined organ matching system (“OMS”) directed to provide for the convenient, immediate, and accurate matching of a donated organ to an appropriate recipient, minimizing down time and the potential of creating “wasted organs” as discussed earlier. Also, proprietary “A-Medicare” algorithms allow for faster and more accurate reading of X-ray studies, MM exams and CT scans, and provide for the ability to listen and send a patient to the correct doctor immediately while also considering genetic analysis and diagnostics to better understand a patient, participant and/or user's possible health risk factors.

“A-Medicare” may be made available in a digital format that may be, for example (but not limitation thereto), any one or more of: a panel, portal, website, and/or app intended to create direct competition between health care providers to earn business from patients, etc. In doing so, “A-Medicare” will also permit patients, participants, users and/or the like to manage doctor's appointments, have their entire medical record available, compare health care service prices to choose the best and most economical for at least the following types of services: health care insurance (e.g., apply for and switch between various private party health insurances through the “A-Medicare” platform and/or obtain health insurance coverage through “A-Medicare”), hospital services/surgeries/ER visits, medications, doctor specialists, and pharmacies (including near by a home/zip code, city and nationwide).

By way of example, “A-Medicare” also provides for a recycling program for unused medication that will reward participants, e.g., by providing credits that may be tied to currency, who maintain a healthy lifestyle and also recycle unused medication to, for example (but not limitation thereto) their local pharmacy. Also, “A-Medicare” may confidentially share information, data analysis and projections with the US federal and/or state and/or local governments, and also, depending on configuration, to foreign nations, territories and/or jurisdiction pursuant to their particular respective needs.

### System Structure

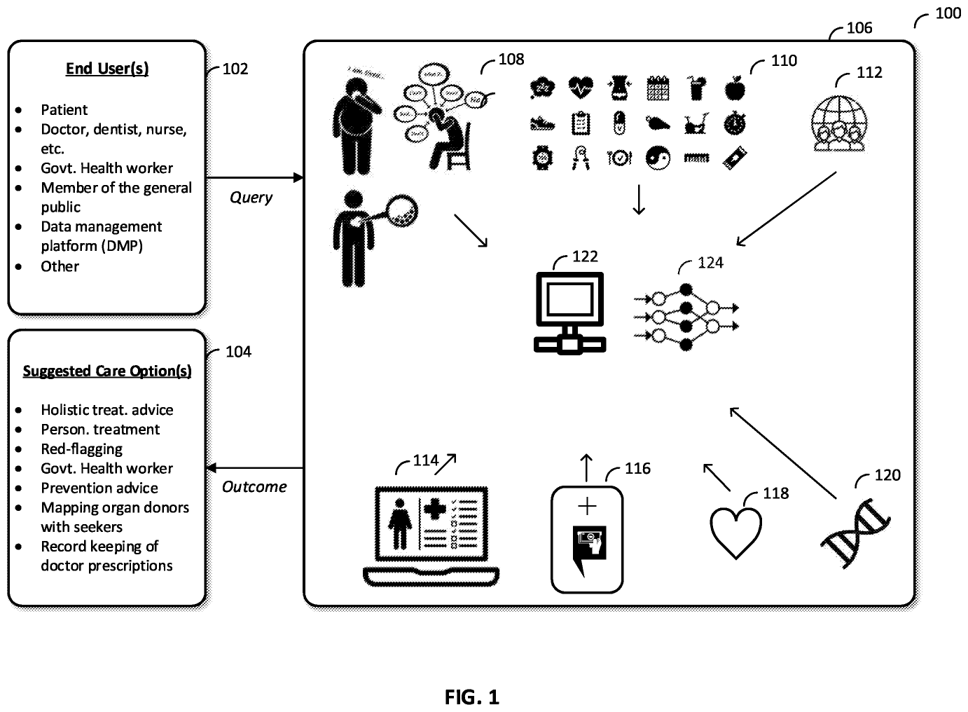

FIG. 1 illustrates an exemplary schematic diagram of an end-user to system to outcome diagram of a virtual intelligent healthcare-related services system directed to improve the effectiveness of providing healthcare services while reducing costs by implementing machine learning, in accordance with an embodiment of the present invention. Referring to the present embodiment, a machine learning system 100 is shown, the machine learning system 100 receiving input from an end user 102 into a healthcare computational module 106 that receives and processes a variety of end user data 108-120 to produce and communicate suggested care options at an outcome module 104. The machine learning system 100 seeks to improve overall effectiveness in providing healthcare related services through enhanced efficiency made possible through technology-driven machine learning capabilities. Those skilled in the art, in light of the teachings of the present invention, and depending upon the needs of the particular application, will readily recognize and appreciate a multiplicity of suitable conventional ways to implement blockchain, AI, machine learning, etc. to enable the machine learning system 100 to produce suggested care options for the end user 102 at the outcome module 104 upon receipt of end user 102 related data.

By way of example and not limitation, end users 102 of the machine learning system 100 may include any one or more qualified participants, including citizens, residents, and/or any other type of lawfully residing alien of a participating nation, e.g., the US, interested in obtaining healthcare-related services and/or insurance coverage through the machine learning system 100, which in one or more embodiments may be a computational and or computer-based algorithmic manifestation of the “A-Medicare”system and/or digital platform introduced earlier. Accordingly, the machine learning system 100 may provide a holistic suite of healthcare related services, including (but not limited to): intake of end user 102 data to yield suggested care options at the outcome module 104.

In one or more embodiments, end users 102 may further and/or alternatively include doctors, dentists, nurses, and/or other healthcare providers, government healthcare workers, any member of the general public, data management platform(s), and/or any other real (e.g., human) and/or digital entity who (or which) may submit a query 126, e.g., a form of inquiry submitted via digital and/or wireless telecommunications means, to the healthcare computational module 106. The query 126 may include specific information regarding any one or more specific data points, including (but not limited to): disease and ailment history 108, lifestyle related information 110, demographics and/or psychological demographics (“psychographics”) related information 112, electronic medical records 114, medical claims 116, treatment history 118 and/or genomics (e.g., inclusive of DNA and/or biomarkers related data) 120.

Such end user data 108-120 is input into a healthcare database 122 that may be at least partially implemented in computer-based hardware, e.g., as executed on one or more processors, and/or stored in or on a virtual network, e.g., at a “cloud”—based location. The healthcare database 122 may at least partially employ machine learning capabilities 124 to automatically compute particularized healthcare recommendations for each end user 102 inputting his or her (or its) respective data into the healthcare computational module 106 of the machine learning system 100. In one or more embodiments, the machine learning system 100 may functionally integrate with any one or more modules, systems, and/or methods of use thereof presented and/or discussed earlier and/or in connection with the FIGs.

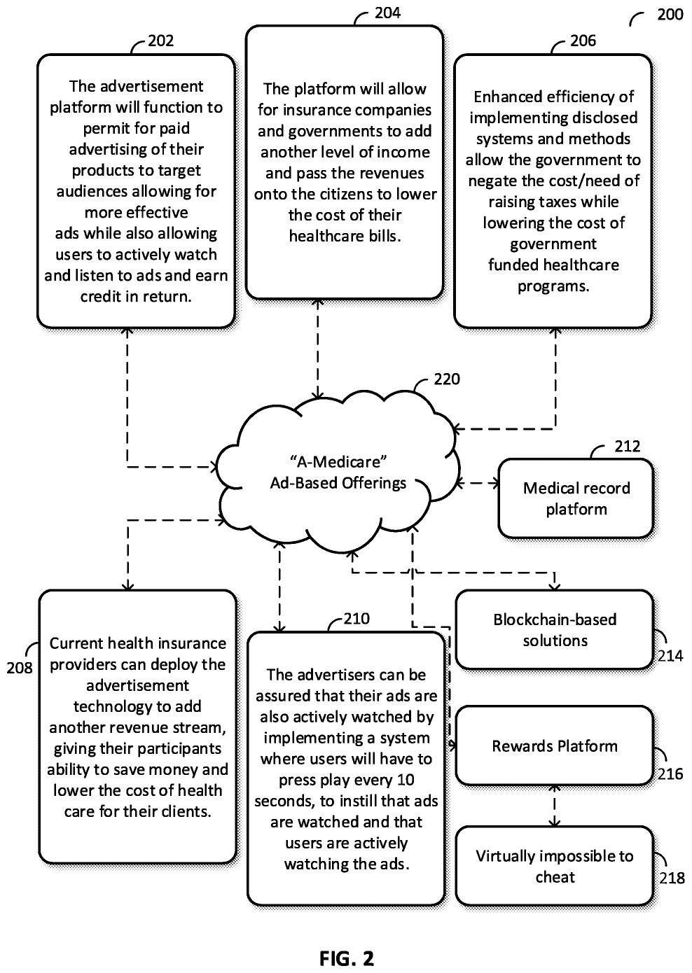

FIG. 2 illustrates an example “cloud”-based system for providing an advertising platform to at least partially generate credits for users who view the advertising, the system utilizing blockchain-based solutions, in accordance with an embodiment of the present invention. Referring to the present embodiment, an advertisement system 200 includes an ad-based offerings module 220 of the “A-Medicare” system and/or digital platform introduced and discussed earlier is shown as stored in a virtual cloud-based location in ongoing electronic communication with various ad-related modules 202-218.

By way of example and not limitation, ad-based offerings module 220 of the “A-Medicare” system may include private-party originated paid advertising content 202 that includes an advertisement platform will function to permit for paid advertising of their products to target audiences allowing for more effective ads while also allowing users to actively watch and listen to ads and earn credit in return. Moreover, ad-based offerings module 220, via an insurance module 204, will allow for insurance companies and governments to add another level of income and pass the revenues onto the citizens to lower the cost of their healthcare bills. Further, integration of ad-based capabilities may allow the ad-based offerings module 220 to improve overall healthcare provision efficiency at a negate raising taxes module 206, where the enhanced efficiency of implementing disclosed systems and methods allow the government to negate the cost/need of raising taxes while lowering the cost of government funded healthcare programs.

Additional beneficial functionality of the ad-based offerings module 220 includes communication and/or functional integration with accommodations for current healthcare and/or health insurance providers at a health insurance provider module 208, where current health insurance providers can deploy the advertisement technology to add another revenue stream, giving their participants ability to save money and lower the cost of health care for their clients. Time increment module 210 may be in communication and/or functionally integrated with the ad-based offerings module 220 such that advertisers can be assured that their ads are actively watched by “A-Medicare” qualified participants by implementing a system where users will have to press play every 10 seconds, to instill that ads are watched and that users are actively watching the ads.

Moreover, the ad-based offerings module 220 communicates and/or may be functionally integrated with a medical record platform 212, that stores personal health related data and/or information particular to individual “A-Medicare” qualified participants, e.g., patients, etc. The medical record platform 212 may store history and documentation for “A-Medicare” qualified participants with the provision for seeking appointments with medical practitioners and streamlining the overall process flow of providing medical diagnosis.

The ad-based offerings module 220 communicates and/or may be functionally integrated with a blockchain-based solutions module 214, that may be immutable and thus ensure “A-Medicare” qualified participants and participating healthcare providers that any and all exchanged data via the ad-based offerings module 220 is protected and safe. Moreover, the blockchain-based solutions module 214 may be configured to provide checks and balances to detect and counteract potential abuses of the “A-Medicare” system and/or digital platform more generally, including protection against improper medical exams, tests, over-prescription of medication and many other use cases that are susceptible to abuse in the medical field.

The ad-based offerings module 220 communicates and/or may be functionally integrated with a rewards platform 216 that may be virtually impossible to cheat, e.g., as denoted by a related anti-cheating module 218, to incentivize qualified “A-Medicare” participants to live a healthy lives by implementing the blockchain-based solutions module 214 to reward users by providing monetary-equivalent credit towards their respective healthcare bills and healthcare premiums for living a healthy and active lifestyle, and will motivate people and help people afford health and/or general wellness programs, as well as for paying for a gym membership, as “A-Medicare” participants will be saving money by using the system and/or digital platform that they would otherwise be paying for healthcare related expenses. Those skilled in the art will appreciate that any one or more of the modules and/or systems 202-218 are provided as an example and that fewer, greater, or other modules and/or systems may be functionally integrated with the ad-based offerings module 220 of the “A-Medicare” system and/or digital platform as shown and described in FIG. 2 without departing from the scope and spirit of the disclosed embodiments. Also, the ad-based offerings module 220 may at least be partially implemented in computer-based hardware, e.g., servers, and/or stored in a non-transitory medium, in addition to a virtual and/or “cloud” based location.

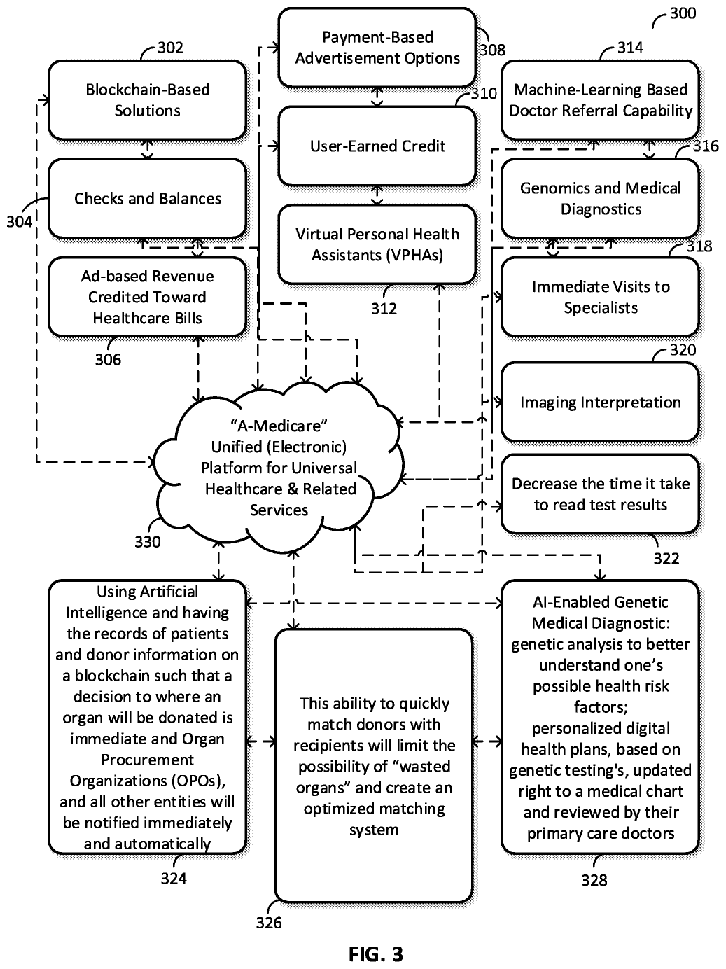

FIG. 3 illustrates an example “cloud”-based system for providing a unified electronic platform for universal healthcare and related services using artificial intelligence (“AI”) to better understand patient health risk factors and to generate personalized digital health plans, in accordance with an embodiment of the present invention. Referring to the present embodiment, a healthcare system 300 includes “A-Medicare”, defined herein and elsewhere as a unified (electronic) platform for universal healthcare and related services 330, that is integrated with and/or functionally communicates various systems and/or modules 302-328 to efficiently and cost-effectively provide healthcare related services.

By way of example and not limitation, the unified (electronic) platform for universal healthcare and related services 330 integrates blockchain-based solutions 302 to provide checks and balances 304 against healthcare provision abuse by providers while also accommodating ad-based revenue that may be credited toward healthcare bills 304. Such ads permit for entities such as businesses, governments and patients participating in the unified (electronic) platform for universal healthcare and related services 330 to receive higher revenues and credit towards their respective health care bills.

Payment-based ad options 308 permit for the at least partial subsidy of healthcare related costs for participants who view such ads and respond to surveys and/or questionnaires, where such results are then forwarded to the respective private entities paying for the ads, etc., to correspondingly yield user-earned credit 310 commensurate with the ads viewed. Such user-earned 310 may be applied to pay for healthcare related services.

The unified (electronic) platform for universal healthcare and related services 330 may also provide for virtual personal health assistants (VPHAs) 312, which are healthcare specific virtual assistants, e.g., chatbots, and combine voice automation and AI to follow voice (or text) commands to provide healthcare-related information and assistance upon user request and/or interaction. VPHAs 312 may be implemented in user-held smartphones and/or devices allowing for unlimited access to VPHAs 312 and if needed, connection directly to a patient or participant's primary care doctor and/or care team. VPHAs 312 may be configured to function to remind a “A-Medicare” patients and/or participants to abide to prescribed and/or issued treatment plans, and may thus provide notifications such as (but not limited to): reminders to take medications, supplements, and make appointments for check-ups. Such capabilities, also allow for immediate responses to patient-initiated questions, anytime/anywhere.

Moreover, the unified (electronic) platform for universal healthcare and related services 330 includes a machine-learning based doctor referral capability 314, related to receiving patient input information to correspondingly direct the patient to the correct doctor immediately, thus decreasing the need for multiple doctor's appointments only to receive the correct referral.

Genomics and medical diagnostics module 316 of the unified (electronic) platform for universal healthcare and related services 330 uses AI to assist in making medical diagnostics more accurate and accessible by using machine learning to predict what will happen based at least partially on detected on symptoms and a patient's genetic makeup. By using basic medical data, genetic testing and machine learning, the genomics and medical diagnostics module 316 may predict months, even years out, an individual's predisposition to certain cancers, metabolic processes and chronic diseases based on detected and/or input medical data. Such functionality and/or capabilities will allow for immediate visits to specialists such as heart health, cancer, and general wellness based on the severity of the illness. Also, the genomics and medical diagnostics module 316 may provide the ability to create personalized health plans, supplement intake programs and/or regimens, and fitness plans based on a patient and/or participant's particular genetic makeup. Patients who are at high risk for specific conditions may receive a free take home wearable monitor to detect, monitor, diagnose and correspondingly alert medical professionals when a medical emergency is occurring. Such AI-enabled genetic and/or medical analysis allows for the better understanding of an “A-Medicare” patient and/or participant's possible health risk factors. Accordingly, personalized digital health plans, based on genetic testing conducted at least in part by the genomics and medical diagnostics module 316 may be updated to a medical chart and reviewed by, for example (but not limitation thereto), a patient's primary care doctor as denoted by a AI-enable genetic medical diagnostic review module 328 to permit for immediate visits to specialists 318, if needed.

The unified (electronic) platform for universal healthcare and related services 330, in one or more embodiments, also incorporates imaging interpretation capabilities 320, which may employ deep learning techniques and/or related categorization technology on very large sets of medical images to create algorithms that allow for faster and more accurate readings of, for example (but not limitation thereto): X-ray studies, MRI exams and CT scans. Such improvements in imaging interpretation, in turn, may decrease the time necessary to read test results 322 to improve healthcare related outcomes for patients who may have life-threatening medical illnesses.

Further, the unified (electronic) platform for universal healthcare and related services 330 may use AI and have the records of patients and donor information on a blockchain, such that a decision to where an organ will be donated is immediate and Organ Procurement Organizations (“OPOs”), and all other entities will be notified immediately and automatically at an organ donation notification module 324. Such an ability as described to quickly match donors 326 with recipients will limit the possibility of “wasted organs” and create an optimized matching system as denoted by an optimization module that may be functionally interconnected with the organ donation notification module and/or the unified (electronic) platform for universal healthcare and related services 330. Those skilled in the art will appreciate that any one or more of the modules and/or systems 302-328 are provided as an example and that fewer, greater, or other modules and/or systems may be functionally integrated with the ad-based offerings module the unified (electronic) platform for universal healthcare and related services 330 as shown and described in FIG. 3 without departing from the scope and spirit of the disclosed embodiments. Also, the unified (electronic) platform for universal healthcare and related services 330 may at least be partially implemented in computer-based hardware, e.g., servers, and/or stored in a non-transitory medium, in addition to a virtual and/or “cloud” based location.

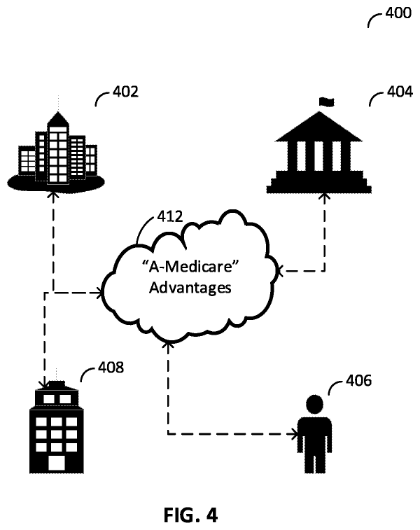

FIG. 4 illustrates an example “cloud”-based system for providing various competitive advantages between different institutions, including private, public and government-owned and/or controlled entities, in accordance with an embodiment of the present invention. Referring to the present embodiment, an infrastructure integration system 400 includes an “A-Medicare” advantages module 412 that may be in electronic communication and/or otherwise functionally integrated with each a private-sector advertising source 402, a government-based agency 404, healthcare providers 408 (e.g., such as private and/or at least partially government funded hospitals), and a patient, participant, treatment candidate, etc., collectively referred to as a user 406.

By way of example and not limitation, the “A-Medicare” will be also able to provide several other services useful to society such as reducing cost for US government and the citizens with Artificial Intelligence (to prevent diseases, to help each citizen to learn about their bodies on what's good or bad for their body/system and more). The government will gain digital control of every health care cost and will be able to supervise and intervene immediately to fix any money waste, health care scams (overbilling) and high costs out of control. The “A-Medicare” advantages module 412 integrates (by way of example and not limitation): machine learning (also ability to alert in advance every citizen in case of any anomalies), blockchain technology (including the capability to connect the right organ donor with recipient nationally and worldwide and track/protect organ transportation), and setting up an international universal database for every citizen where they can also get consultations from doctors worldwide.

In one or more embodiments, the “A-Medicare” advantages module 412 provides a strategic panel system for every user 406 (locked with an assigned ID and Social Security number) where they can manage doctor's appointments, have their entire medical record available, choose the best and most cost-effective solution/option regarding: health care insurance, hospital services/surgeries/ER visits, medications, doctor specialist, and pharmacy. The “A-Medicare” advantages module 412 further permits for the ability to receive user 406 comments and rate for the services received.

The “A-Medicare” advantages module 412 also provides for each user 406 to be able to watch ads that will help them to pay their health care premiums (e.g., equating to about $350 each month).

By way of example and not limitation, the “A-Medicate” advantages module includes strategic advertising capabilities and insider collaboration between corporations and US Government will help to effectively reduce health care costs in a short/medium timeframe and will help citizens in, for example (but not limitation thereto) medium and lower classes to get obtain health care coverage. The US government may collect any necessary funds from “A-Medicare (e.g., minus 20% of the revenues).

By way of example and not limitation, the “A-Medicare” 412 advantages module includes a viewers' attention platform that can enable the vital participants in the ad ecosystem—namely advertisers (insurance companies, etc.), publishers (online platforms) and browser users—to participate in a new business model that cuts out excess intermediation between publishers and advertisers. The platform creates a new measure of value in the advertising world, “consumer attention,” rather than the current measure of meaningless, unverifiable views or clicks on a webpage. User will have to actively watch each add that benefits the advertisers.

To achieve this, the “A-Medicare” 412 advantages module has a system that verifies a person's phone, IPAD or other similar devices though knowing their IP address in relation to their location and disable the use of VPS so it cannot be outsourced. Allowing Advertiser to be rest assured their ad is being watch by the intended demographic.

Including a protocol in place that make the participant have the phone volume to an acceptable level as well as that (under discussion) they have to press play every 10 seconds. This time frame will not allow one to switch apps due to having to have the music play only if the app if the primary app being used and active participation though having to press play.

Special programs and rewards credit in the A-Medicare platform/portal provide that each user 406 that keeps an healthy lifestyle and recycles unused medication to their local pharmacy and give and make those medication for a 50% of the original price (25% to the pharmacies so they can make a small profit for processing and cooperate with the program, 20% to A-Medicare and 5% back to the citizen panel as incentive);

A strategic advertising system and insider collaboration between corporations and US Government will help to effectively reduce health care costs in a short/medium timeframe and will help citizens especially mediums and low class to get proper health care coverage.

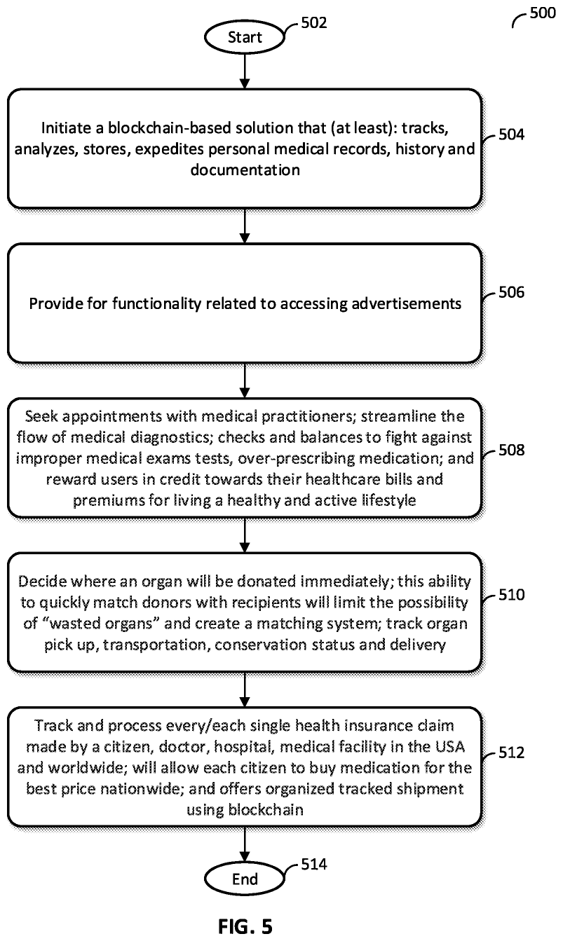

FIG. 5 illustrates a flowchart of an exemplary method that uses blockchain technologies to track, analyze, store, and expedite personal medical records, history and documentation, in accordance with an embodiment of the present invention. Referring to the present embodiment, a method 500 begins at a start operation 502. A blockchain-based solution is initiated at operation 504 that (at least): tracks, analyzes, stores, expedites personal medical records, history and documentation. Functionality is provided for at operation 506 related to accessing advertisements. Appointments may be sought in operation 506 with medical practitioners; e.g., to streamline the flow of medical diagnostics; provide checks and balances to fight against improper medical exams tests, over-prescribing medication; and reward users in credit towards their healthcare bills and premiums for living a healthy and active lifestyle. At operation 510, a decision may be made regarding where an organ will be donated immediately; this ability to quickly match donors with recipients will limit the possibility of “wasted organs” and create a matching system; track organ pick up, transportation, conservation status and delivery. Operation 512 permits for the tracking and processing every/each single health insurance claim made by a citizen, doctor, hospital, medical facility in the USA and worldwide; will allow each citizen to buy medication for the best price nationwide; and offers organized tracked shipment using blockchain prior to ending at operation 514.

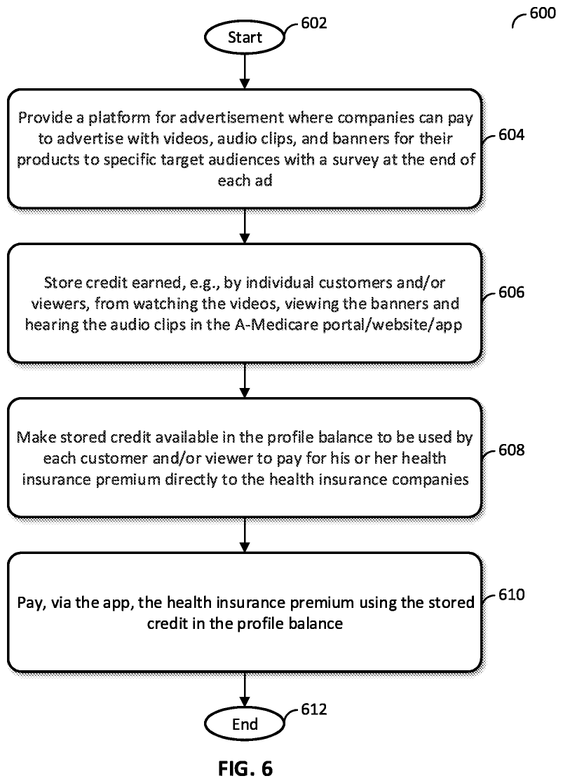

FIG. 6 illustrates a flowchart of an exemplary method to provide a platform for advertisement (“ad”) where companies can pay to advertise with videos, audio clips, and banners for their products to specific target audiences with a survey at the end of each ad, in accordance with an embodiment of the present invention. Referring to the present embodiment, a method 600 begins with a start operation 602. A platform is provided at an operation 604 for advertisement where companies can pay to advertise with videos, audio clips, and banners for their products to specific target audiences with a survey at the end of each ad. Store credit earned at an operation 606, e.g., by individual customers and/or viewers, from watching the videos, viewing the banners and hearing the audio clips in the “A-Medicare” portal/website/app. Stored credit is made available at an operation 608 in the profile balance to be used by each customer and/or viewer to pay for his or her health insurance premium directly to the health insurance companies. Payment is provided at an operation 610, via the app, the health insurance premium using the stored credit in the profile balance prior to an end operation 612.

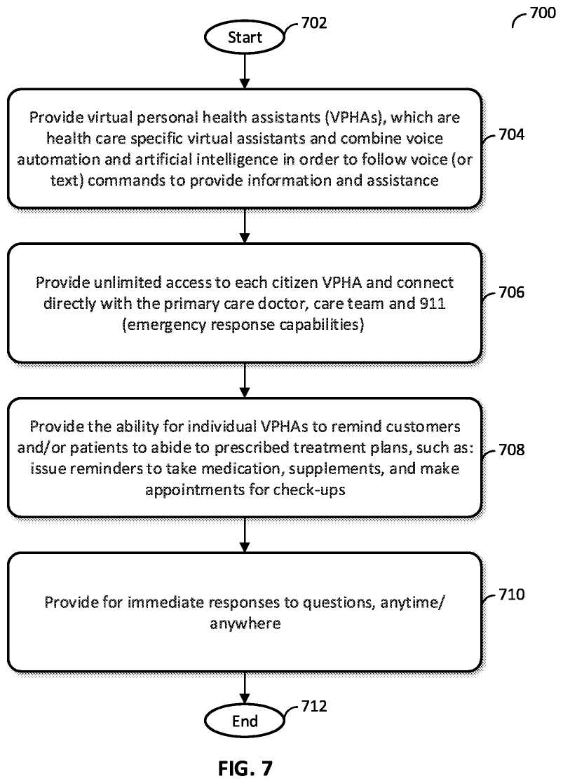

FIG. 7 illustrates a flowchart of an exemplary method to provide virtual personal health assistants (VPHAs), which are health care specific virtual assistants and combine voice automation and artificial intelligence in order to follow voice (or text) commands to provide information and assistance, in accordance with an embodiment of the present invention. Referring to the present embodiment, a method 700 begins at a start operation 702. Virtual personal health assistants (VPHAs) are provided at an operation 702, which are health care specific virtual assistants and combine voice automation and artificial intelligence in order to follow voice (or text) commands to provide information and assistance. Unlimited access is provided at an operation 706 to each citizen VPHA and connects directly with the primary care doctor, care team and 911 (emergency response capabilities). The ability for individual VPHAs to remind customers and/or patients to abide to prescribed treatment plans, such as: issue reminders to take medication, supplements, and make appointments for check-ups is provided for in operation 708. Immediate responses to questions, anytime/anywhere are provided for in operation 710 prior to an end operation 712. At an organizational level, checks and balances are included and not limited to screening the VPHA to user to user, user to provider and provider to provider by screening all participants.

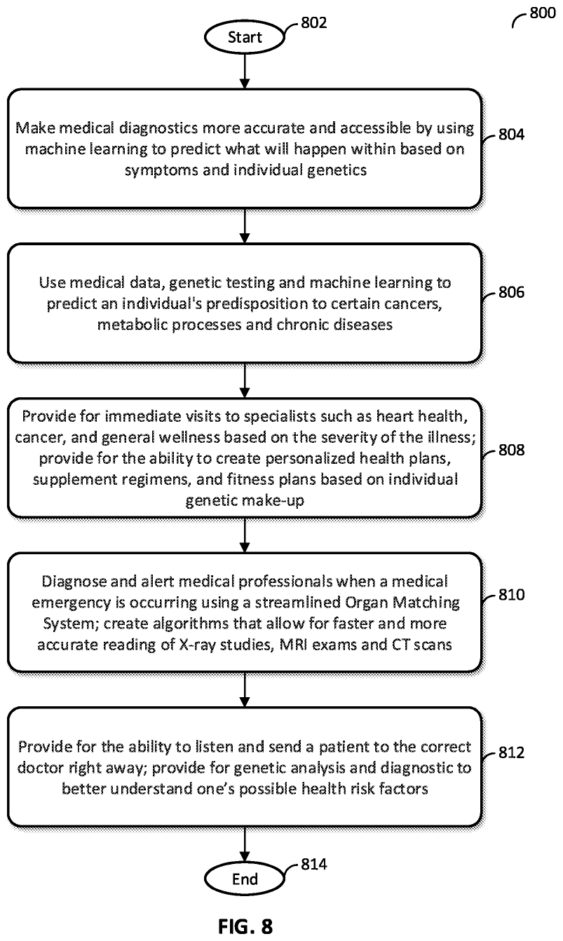

FIG. 8 illustrates a flowchart of an exemplary method to make medical diagnostics more accurate and accessible by using machine learning to predict what will happen within based on symptoms and individual genetics, in accordance with an embodiment of the present invention. Referring to the present embodiment, a method 800 is shown that begins at a start operation 802. Medical diagnostics more accurate and accessible by using machine learning, at an operation 804, to predict what will happen within based on symptoms and individual genetics. Medical data stored in memory including but not limited to 5D storage is used at an operation 806, genetic testing and machine learning to predict an individual's predisposition to certain cancers, metabolic processes and chronic diseases. Immediate visits are provided for in an operation 808 to specialists such as heart health, cancer, and general wellness based on the severity of the illness; provide for the ability to create personalized health plans, supplement regimens, and fitness plans based on individual genetic makeup. An operation 810 involves the diagnosis and alerting of medical professionals when a medical emergency is occurring using a streamlined Organ Matching System, e.g., also creating algorithms that allow for faster and more accurate reading of X-ray studies, MM exams and CT scans. The ability to listen and send a patient to the correct doctor right away is provided for at an operation 812; e.g., including providing for genetic analysis and diagnostic to better understand one's possible health risk factors prior to an end operation 814.

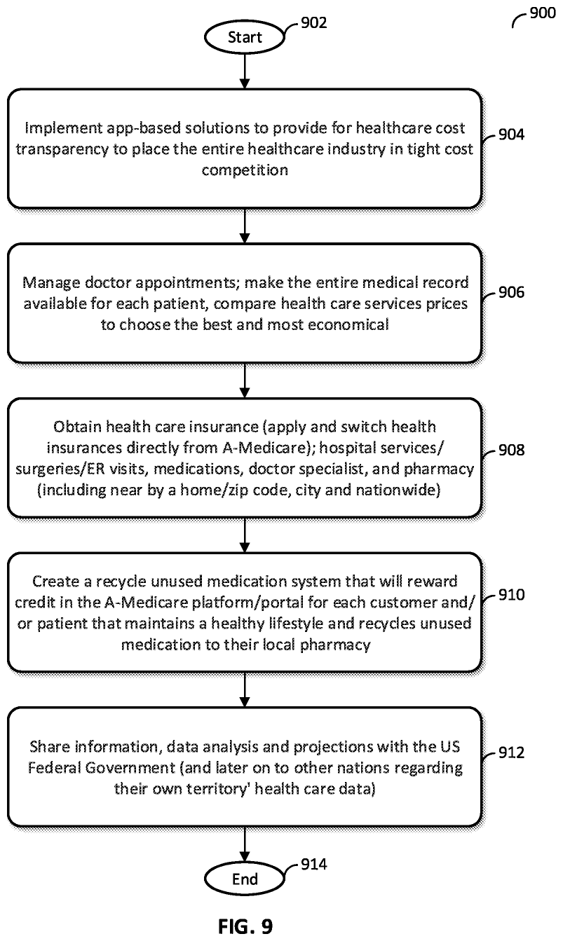

FIG. 9 illustrates a flowchart of an exemplary method to implement app-based solutions to provide for healthcare cost transparency to place the entire healthcare industry in tight cost competition, in accordance with an embodiment of the present invention. Referring to the present embodiment, method 900 begins at a start operation 902. App-based solutions are implemented in an operation 904 to provide for healthcare cost transparency to place the entire healthcare industry in tight cost competition. Doctor appointments are managed at an operation 906; e.g., including making the entire medical record available for each patient, compare health care services prices to choose the best and most economical. Health care insurance is obtained at an operation 908 (apply and switch health insurances directly from A-Medicare); hospital services/surgeries/ER visits, medications, doctor specialist, and pharmacy (including near by a home/zip code, city and nationwide). A recycle unused medication system operation 910 reward credit in the “A-Medicare” platform/portal for each customer and/or patient that maintains a healthy lifestyle and recycles unused medication to their local pharmacy. Information, data analysis and projections are shared at an operation 912 with the US Federal Government (and later on to other nations regarding their own health care data) prior to an end operation 914.

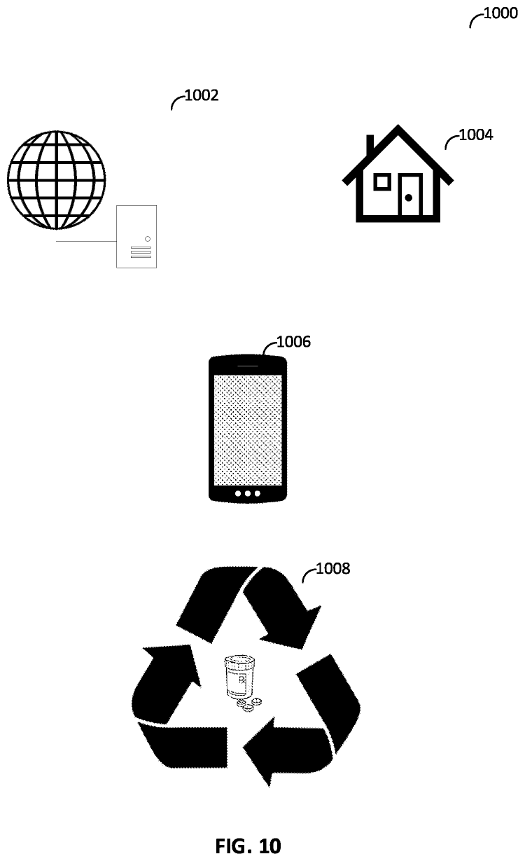

FIG. 10 illustrates electronic communication mediums and/or devices related to increasing competition in the entire healthcare industry and/or for recycling unused medication, in accordance with an embodiment of the present invention. Referring to the present embodiment, a system 1000 is shown allowing for the presence of the “A-Medicare” system and/or digital platform as disclosed in other embodiments to be implemented as a website 1002 accessible via the Internet. Customers may register from a home-based location 1004 and/or remotely access “A-Medicare” capabilities via a smartphone and/or device 1006. Medication recycling capabilities 1008 are integrated throughout to yield customer credit and/or benefit.

Those skilled in the art will readily recognize, in light of and in accordance with the teachings of the present invention, that any of the foregoing steps and/or system modules may be suitably replaced, reordered, removed and additional steps and/or system modules may be inserted depending upon the needs of the particular application, and that the systems of the foregoing embodiments may be implemented using any of a wide variety of suitable processes and system modules, and is not limited to any particular computer hardware, software, middleware, firmware, microcode and the like.

For any method steps described in the present application that can be carried out on a computing machine, a typical computer system can, when appropriately configured or designed, serve as a computer system in which those aspects of the invention may be embodied. Such computers referenced and/or described in this disclosure may be any kind of computer, either general purpose, or some specific purpose computer such as, but not limited to, a workstation, a mainframe, GPU, ASIC, etc. The programs may be written in C, or Java, Brew or any other suitable programming language. The programs may be resident on a storage medium, e.g., magnetic or optical, e.g., without limitation, the computer hard drive, a removable disk or media such as, without limitation, a memory stick or SD media, or other removable medium. The programs may also be run over a network, for example, with a server or other machine sending signals to the local machine, which allows the local machine to carry out the operations described herein.

System Integration with Client/Server Systems

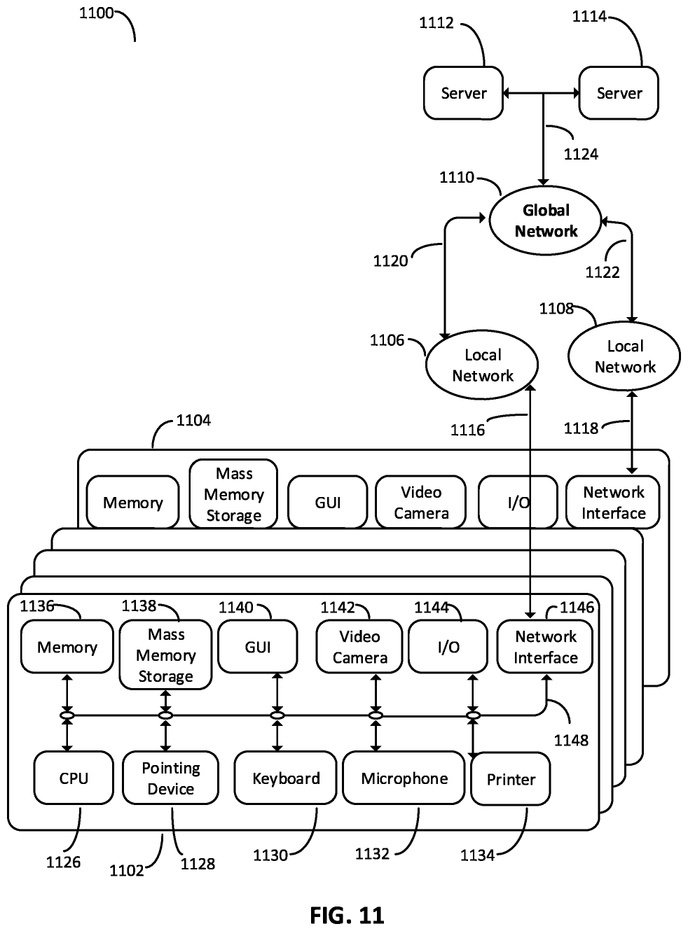

FIG. 11 is a block diagram depicting an exemplary client/server system which may be used by an exemplary web-enabled/networked A-Medicare virtual transaction system embodiment of the present invention. In one embodiment, A-Medicare cryptocurrency provides users a mechanism to interact in the virtual environment by transacting with other users across networks or the Internet. Cryptocurrency may comprise a form of digital asset or virtual currency that is distributed across a large number of computers or nodes, and may be secured by cryptography to conduct financial transactions which may make it substantially resistant to counterfeiting or double-spending. Cryptocurrencies are generally decentralized networks based on blockchain technology, which is a distributed ledger enforced by a network of computers, referred to as nodes. Cryptocurrencies may leverage blockchain technology to gain decentralization, transparency, immutability, and substantially ensuring the integrity of transactional data. Blockchain may include a shared, trusted, public ledger of transactions, that everyone may inspect but which no one controls. The blockchain may include a distributed database that maintains a continuously growing list of transaction data records written into blocks that are interconnected together and cryptographically secured from tampering and revision. Once a block reaches a certain number of approved transactions then a new block is formed.

In one embodiment, A-Medicare communication system 1100 includes a multiplicity of nodes or clients with a sampling of clients denoted as a client/node 1102 and a client/node 1104, a multiplicity of local networks with a sampling of networks denoted as a local network 1106 and a local network 1108, a global network 1110 and a multiplicity of servers with a sampling of servers denoted as a server 1112 and a server 1114. Each node may have a copy of the Blockchain.

Client/Node 1102 may communicate bi-directionally with local network 1106 via a communication channel 1116. Client/Node 1104 may communicate bi-directionally with local network 1108 via a communication channel 1118. Local network 1106 may communicate bi-directionally with global network 1110 via a communication channel 1120. Local network 1108 may communicate bi-directionally with global network 1110 via a communication channel 1122. Global network 1110 may communicate bi-directionally with server 1112 and server 1114 via a communication channel 1124. Server 1112 and server 1114 may communicate bi-directionally with each other via communication channel 1124. Furthermore, clients/nodes 1102, 1104, local networks 1106, 1108, global network 1110 and servers 1112, 1114 may each communicate bi-directionally with each other.

In one embodiment, global network 1110 may operate as the Internet. It will be understood by those skilled in the art that communication system 1100 may take many different forms. Non-limiting examples of forms for communication system 1100 include local area networks (LANs), wide area networks (WANs), wired telephone networks, wireless networks, or any other network supporting data communication between respective entities.

Clients/Nodes 1102 and 1104 may take many different forms. Non-limiting examples of clients 1102 and 1104 include personal computers, personal digital assistants (PDAs), cellular phones and smartphones. By spreading operations across a network of clients/nodes 1102 and 1104, blockchain may allow cryptocurrencies to operate without the need for a central authority. The operation may reduce risks and may eliminate many processing and transaction fees. When the network of clients or nodes 1102 and 1104 validate a transaction by majority consensus, the transaction may be permanently written into a block of the blockchain. Otherwise, the transaction may be rejected and does not go through. Transactions that have been included in the blockchain may be considered as valid and final.

Client 1102 includes a CPU 1126, a pointing device 1128, a keyboard 1130, a microphone 1132, a printer 1134, a memory 1136, a mass memory storage 1138, a GUI 1140, a video camera 1142, an input/output interface 1144 and a network interface 1146.

CPU 1126, pointing device 1128, keyboard 1130, microphone 1132, printer 1134, memory 1136, mass memory storage 1138, GUI 1140, video camera 1142, input/output interface 1144 and network interface 1146 may communicate in a unidirectional manner or a bi-directional manner with each other via a communication channel 1148. Communication channel 1148 may be configured as a single communication channel or a multiplicity of communication channels.

CPU 1126 may be comprised of a single processor or multiple processors. CPU 1126 may be of various types including micro-controllers (e.g., with embedded RAM/ROM) and microprocessors such as programmable devices (e.g., RISC or SISC based, or CPLDs and FPGAs) and devices not capable of being programmed such as gate array ASICs (Application Specific Integrated Circuits) or general-purpose microprocessors.

As is well known in the art, memory 1136 is used typically to transfer data and instructions to CPU 1126 in a bi-directional manner. Memory 1136, as discussed previously, may include any suitable computer-readable media, intended for data storage, such as those described above excluding any wired or wireless transmissions unless specifically noted. Mass memory storage 1138 may also be coupled bi-directionally to CPU 1126 and provides additional data storage capacity and may include any of the computer-readable media described above. Mass memory storage 1138 may be used to store programs, data and the like and is typically a secondary storage medium such as a hard disk. It will be appreciated that the information retained within mass memory storage 1138, may, in appropriate cases, be incorporated in standard fashion as part of memory 1136 as virtual memory.

CPU 1126 may be coupled to GUI 1140. GUI 1140 enables a user to view the operation of computer operating system and software. CPU 1126 may be coupled to pointing device 1128. Non-limiting examples of pointing device 1128 include computer mouse, trackball and touchpad. Pointing device 1128 enables a user with the capability to maneuver a computer cursor about the viewing area of GUI 1140 and select areas or features in the viewing area of GUI 1140. CPU 1126 may be coupled to keyboard 1130. Keyboard 1130 enables a user with the capability to input alphanumeric textual information to CPU 1126. CPU 1126 may be coupled to microphone 1132. Microphone 1132 enables audio produced by a user to be recorded, processed and communicated by CPU 1126. CPU 1126 may be connected to printer 1134. Printer 1134 enables a user with the capability to print information to a sheet of paper. CPU 1126 may be connected to video camera 1142. Video camera 1142 enables video produced or captured by user to be recorded, processed and communicated by CPU 1126.

CPU 1126 may also be coupled to input/output interface 1144 that connects to one or more input/output devices such as such as CD-ROM, video monitors, track balls, mice, keyboards, microphones, touch-sensitive displays, transducer card readers, magnetic or paper tape readers, tablets, styluses, voice or handwriting recognizers, or other well-known input devices such as, of course, other computers.

Finally, CPU 1126 optionally may be coupled to network interface 1146 which enables communication with an external device such as a database or a computer or telecommunications or internet network using an external connection shown generally as communication channel 1116, which may be implemented as a hardwired or wireless communications link using suitable conventional technologies. With such a connection, CPU 1126 might receive information from the network, or might output information to a network in the course of performing the method steps described in the teachings of the present invention.

System Integration with Web-Enablement and/or Networking

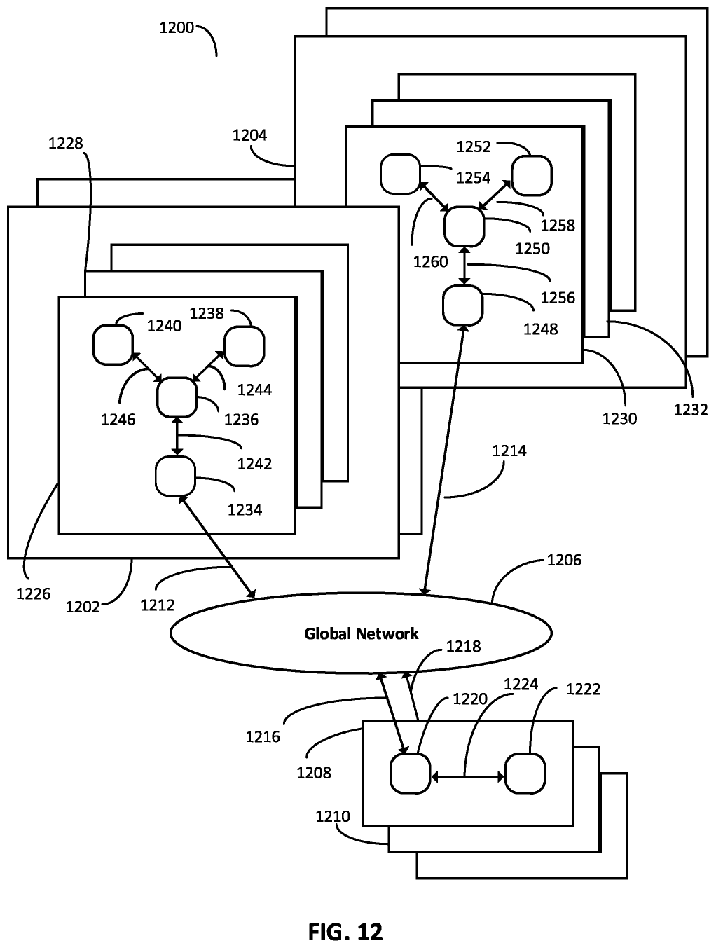

FIG. 12 illustrates a block diagram depicting a conventional client/server communication system, which may be used by an exemplary web-enabled/networked A-Medicare virtual transaction system embodiment of the present invention.

An exemplary A-Medicare communication system 1200 may include a multiplicity of networked regions with a sampling of regions denoted as a network region 1202 and a network region 1204, a global network 1206 and a multiplicity of servers with a sampling of servers denoted as a server device 1208 and a server device 1210.

Network region 1202 and network region 1204 may operate to represent a network contained within a geographical area or region. Non-limiting examples of representations for the geographical areas for the networked regions may include postal zip codes, telephone area codes, states, counties, cities and countries. Elements within network region 1202 and 1204 may operate to communicate with external elements within other networked regions or within elements contained within the same network region.

In some implementations, global network 1206 may operate as the Internet. It will be understood by those skilled in the art that communication system 1200 may take many different forms. Non-limiting examples of forms for communication system 1200 include local area networks (LANs), wide area networks (WANs), wired telephone networks, cellular telephone networks or any other network supporting data communication between respective entities via hardwired or wireless communication networks. Global network 1206 may operate to transfer information between the various networked elements. In some embodiments, blockchain protocol may operate on top of the Internet, on a P2P network of nodes or clients that all run the blockchain protocol and hold an identical copy of the ledger of transactions, enabling P2P value transactions through machine consensus.

Server device 1208 and server device 1210 may operate to execute software instructions, store information, support database operations and communicate with other networked elements. Non-limiting examples of software and scripting languages which may be executed on server device 1208 and server device 1210 include C, C++, C# and Java.

Network region 1202 may operate to communicate bi-directionally with global network 1206 via a communication channel 1212. Network region 1204 may operate to communicate bi-directionally with global network 1206 via a communication channel 1214. Server device 1208 may operate to communicate bi-directionally with global network 1206 via a communication channel 1216. Server device 1210 may operate to communicate bi-directionally with global network 1206 via a communication channel 1218. Network region 1202 and 1204, global network 1206 and server devices 1208 and 1210 may operate to communicate with each other and with every other networked device located within communication system 1200.

Server device 1208 includes a networking device 1220 and a server 1222. Networking device 1220 may operate to communicate bi-directionally with global network 1206 via communication channel 1216 and with server 1222 via a communication channel 1224. Server 1222 may operate to execute software instructions and store information.

Network region 1202 includes a multiplicity of nodes or clients with a sampling denoted as a node 1226 and a node 1228. Node 1226 includes a networking device 1234, a processor 1236, a GUI 1238 and an interface device 1240. Non-limiting examples of devices for GUI 1238 include monitors, televisions, cellular telephones, smartphones and PDAs (Personal Digital Assistants). Non-limiting examples of interface device 1240 include pointing device, mouse, trackball, scanner and printer. Networking device 1234 may communicate bi-directionally with global network 1206 via communication channel 1212 and with processor 1236 via a communication channel 1242. GUI 1238 may receive information from processor 1236 via a communication channel 1244 for presentation to a user for viewing. Interface device 1240 may operate to send control information to processor 1236 and to receive information from processor 1236 via a communication channel 1246. Network region 1204 includes a multiplicity of nodes with a sampling denoted as a node 1230 and a node 1232. Node 1230 includes a networking device 1248, a processor 1250, a GUI 1252 and an interface device 1254. Non-limiting examples of devices for GUI 1238 include monitors, televisions, cellular telephones, smartphones and PDAs (Personal Digital Assistants). Non-limiting examples of interface device 1240 include pointing devices, mousse, trackballs, scanners and printers. Networking device 1248 may communicate bi-directionally with global network 1206 via communication channel 1214 and with processor 1250 via a communication channel 1256. GUI 1252 may receive information from processor 1250 via a communication channel 1258 for presentation to a user for viewing. Interface device 1254 may operate to send control information to processor 1250 and to receive information from processor 1250 via a communication channel 1260.

For example, consider the case where a user interfacing with node 1226 may want to execute a networked virtual currency transaction. A user may enter the public key or IP (Internet Protocol) address for the cryptocurrency wallet using interface device 1240. The public key or IP address information may be communicated to processor 1236 via communication channel 1246. Processor 1236 may then communicate the public key or IP address information to networking device 1234 via communication channel 1242. Networking device 1234 may then communicate the public key or IP address information to global network 1206 via communication channel 1212. Global network 1206 may then communicate the public key or IP address information to networking device 1220 of server device 1208 via communication channel 1216. Networking device 1220 may then communicate the public key or IP address information to server 1222 via communication channel 1224. Server 1222 may receive the public key or IP address information and after processing the public key or IP address information may communicate return information to networking device 1220 via communication channel 1224. The return information may include but not limited to cryptocurrencies, diagnosis, treatment, prescription, etc. Networking device 1220 may communicate the return information to global network 1206 via communication channel 1216. Global network 1206 may communicate the return information to networking device 1234 via communication channel 1212. Networking device 1234 may communicate the return information to processor 1236 via communication channel 1242. Processor 1236 may communicate the return information to GUI 1238 via communication channel 1244. User may then view the return information on GUI 1238. If the user is a healthcare provider, the user may be viewing cryptocurrencies as payment for service. If the user is a patient, the user may be viewing diagnosis, treatment, and/or prescription.

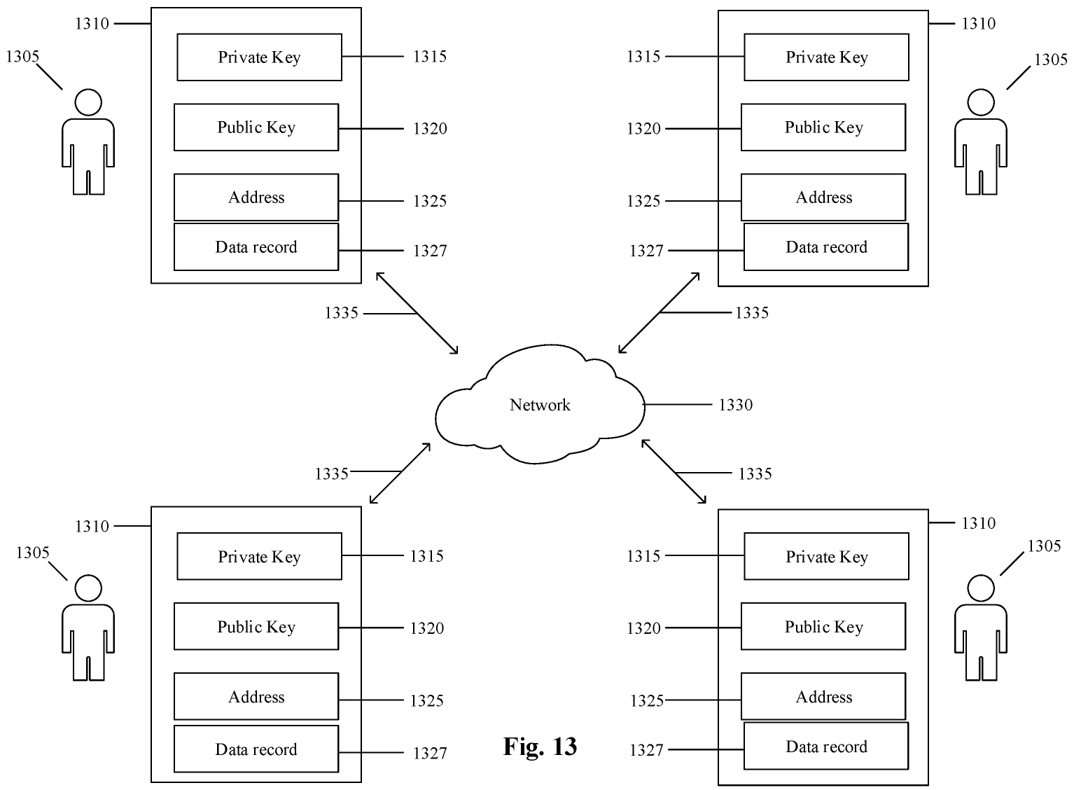

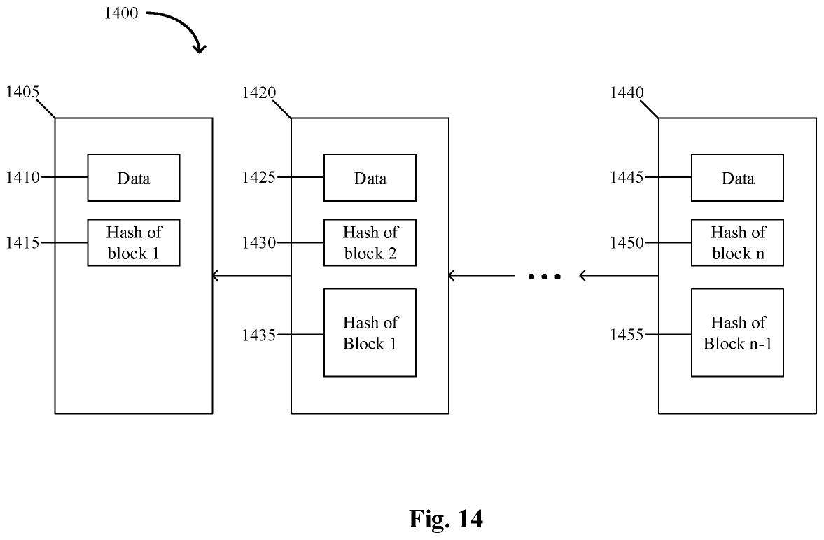

FIG. 13 illustrates an exemplary A-Medicare virtual currency transaction system with A-Medicare cryptocurrency wallets, in accordance with an embodiment of the present invention. The virtual currency transaction system may comprise A-Medicare software related application running A-Medicare wallets. A-Medicare wallet 1310 may include but not limited to, mobile phone-based wallets, desktop-based wallets, web-based wallets, hardware-based wallets, third-party wallet, crystal-based wallets, etc. which facilitates, but not limited to, transactions, ownership tracking of cryptocurrencies, and instant payments. Users 1305 may be connected to network 1330 via network connection 1335 as exemplified in FIGS. 11 and 12. Network connection 1335 may be any type of network connection known in the art, such as, but not limited to, Wireless Fidelity (WiFi), broadband, cellular networks, virtual private networks (VPN), etc. It should be appreciated by one skilled in the art that users 1305 are not limited to individuals or payors, and may be, for example, without limitation, recipients such as health care providers, insurance companies, corporations, government entities, etc. Each of users 1305 may be associated with A-Medicare wallet 1310. A-Medicare wallet 1310 may include data record 1327 containing information for storing cryptocurrency, amount of stored cryptocurrency, as well as sending, and receiving cryptocurrency to and from users 1305. Each wallet may include one or more private keys 1315, one or more public keys 1320, one or more addresses 1325, and one or more data 1327. In one embodiment, the public and private keys may not leave the wallet. The keys may be encrypted. One or more data record 1327 may comprise cryptocurrency and/or a blockchain 1400 (see FIG. 14). Public key 1320 may contain an address of user 1305 sending the public key 1320.

Address 1325 may serve as the location of A-Medicare wallet 1310 which is published to the network/internet and to which other wallet may send or receive cryptocurrencies. Address 1325 may comprise of randomly generated strings of case-sensitive letters, numbers, and/or special characters and may be derived from public key 1320 by application of a hash function. A hash is a string of numbers and letters, produced by hash functions. A hash function is a mathematical function that takes a variable number of characters and converts it into a string with a fixed number of characters. Even a small change in a string creates a completely new hash. In one embodiment, the address is a hashed version of the public key. For instance, the public key may be used to ensure ownership of the wallet that may send or receive cryptocurrency. Public key 1320 may be mathematically derived from private key 1315. Public key 1320 may be digitally signed by private key 1315. To sign public key 1320, a signing software creates a hash of public key 1320. Private key 1315 is then used to encrypt the hash. The encrypted hash, plus the hashing algorithm used, is the digital signature. In addition to the public and private key pairs and address, the wallet may store a log of all incoming and outgoing transactions. Private key 1315 and public key 1320 may be generated by any means known in the art, such as, but not limited to, elliptic curve digital signature algorithm (ECDSA), Rivest-Shamir-Adleman (RSA), etc. Address 1325 may be generated from public key 1320 by any means known in the art, such as, but not limited to, Secure Hash 256 (SHA256), message-digest (MD5), etc. Once a public key 1320 is generated, address 1325 may be shared with another one of users 1305 so that the another one of users 1305 may be able to validate and send cryptocurrency to the owner of address 1320. A-Medicare wallet 1310 may be used on a variety of different platforms, and may be, for example, without limitation, mobile application based, browser based, desktop application based, third party based, etc. While the cryptocurrency associated with A-Medicare wallet 1310 may be used for medical related expenses, it should be appreciated by one skilled in the art that cryptocurrency may be used for a variety of different transactions, such as, but not limited to, paying bills, purchasing commodities, transferring money, receiving financial assistance from a government agency, etc. Transactions may be sent by, for example, without limitation, short-wave radio, photonic beam, sound waves, short waves, Bluetooth, WiFi, etc. By utilizing short wave frequencies, the A-Medicare crypto currency may be transmitted and received without using the traditional method (e.g., blockchain). Photonic beams (of light) may send and receive a transaction providing a sending and receiving device. Photon is the smallest unit of light but each photon particle may contain more information than the largest computer available on earth. Additionally, A-Medicare wallet 1310 may be a crystal wallet, and may be used to interact with other types of cryptocurrencies in the market. The crystal-based wallet may include a 5D data storage element storing a vast amount of data within a crystalline structure. The hardware-based wallet may comprise of a substantial amount of memory and may be used just like a USB flash memory, except the memory may be used to store, but not limited to, the address, the public and private keys, a blockchain with continuously growing blocks, etc.

In an embodiment, to conduct a virtual currency transaction with A-Medicare wallet 1310, a health care provider 1305 provides a public key to a recipient node, for instance, referring to FIG. 11, node 1102 1104. The public key may be provided to the recipient node by scanning a QR code or a barcode. Alternatively, the public key may be provided from a secured storage location of the recipient node. In turn, the recipient node broadcasts the public key across the network or Internet, for instance network 1106 1110. Although all the nodes in the network or Internet can see the public key, only the patient wallet having an address corresponding to the public key may be able to associate the public key with a private key of the patient wallet. The public key is a hash of the address of a patient wallet where cryptocurrencies are withdrawn from. Once the public key is validated to correspond to the patient's wallet private key, a predetermined amount of cryptocurrency is deducted from the funds deposited in the patient's wallet and delivered to the recipient's wallet. A payment message from the patient's wallet may be transmitted across the network or Internet 1106 1110 (FIG. 11) to be received by recipient wallet 1305.

In one embodiment, the public key may include an encrypted information which a corresponding private key may be able to decrypt. In addition, a password may be provided by the patient as a requirement before releasing any amount of funds. In another embodiment, the public key may additionally be digitally signed by the corresponding private key. To digitally sign the public key, a signing software creates a hash of the public key. Patient wallet private key is then used to encrypt the hash. The encrypted hash, plus the hashing algorithm used in encrypting the hash, is the digital signature. Once validation is confirmed, the recipient receives, for example, a predetermined amount of crypto currency from the patients' wallet. The recipient may deposit the received crypto currencies into the health care provider wallet. Alternatively, if the recipient is a patient, the recipient may receive, for example, a diagnosis and/or a treatment performed by the healthcare provider.

FIG. 14 illustrates an exemplary blockchain, in accordance with an embodiment of the present invention. Blockchain 1400 may be used for various processes related to healthcare, such as, but not limited to, storing confidential patient information, healthcare related cryptocurrency, all incoming and outgoing transactions, fund transfer, etc. Blockchain 1400 may include a distributed database that maintains a continuously growing list of transaction data records written into blocks that are interconnected together and cryptographically secured from tampering and revision. Once a block reaches a certain number of approved transactions then a new block is formed. Blockchain 1400 may comprise multiple blocks, such as, without limitation, first block 1405, second block 1420, nth block 1440, etc. Transactions are entered into a block in the order in which they are received, for example, first block 1405. Once a block reaches a predetermined number of approved transactions then a new block is formed, for example, second block 1420 and so on. As will be appreciated by one skilled in the art, there may be any number of blocks in blockchain 1400. First block 1405 may contain, but not limited to, data 1410 and hash of block 1 1415. Any hashing algorithm may be used to compute the hashes of blockchain 1400, such as, but not limited to, SHA256, MD5, etc. First block 1405 may be different than subsequent blocks because first block 1405 may not contain a reference to a hash of a different block. Each succeeding block may contain its own data, a hash of itself, and a hash of the previous block. For example, second block 1420 contains second data 1425, hash of (itself) block 2 1430, and hash of block 1 1435. Similarly, nth block 1440 may contain nth data 1445, hash of block n 1450, and hash of block n−1 1455. Each block refers to the previous block and together make the Blockchain. Each block may contain a record and address of A-Medicare wallet 1310. Each transaction may generate a hash. A small change in a transaction may create a completely new hash. Blockchain 1400 may be a distributed, peer to peer (P2P) blockchain, using validation techniques such as, but not limited to, proof-of-work, etc. The nodes may check to ensure a transaction has not been changed by inspecting the hash. If a transaction is approved by a majority of the nodes then the transaction is recorded into a block.

In one embodiment, health care providers may leverage blockchain 1400 to securely store patients' medical records. Referring to FIGS. 3 and 5, transactions may include blockchain-based solutions 302 (see FIG. 3) 504 (FIG. 5). A medical record may include, for example, patient name, date of birth, diagnosis, treatment, date of doctor's visit, result of treatment, etc. When a patient medical record is generated, the medical record may be broadcast to other nodes for verification. When the medical record is certified to be true and correct, the medical record may be signed, dated and time stamped, then written into a data portion of a block. If it is a new patient, then the medical record performed on the patient on that particular time is written into first block 1405. Patient medical records in a blockchain may be entered in the order in which they occurred. Once a medical record is verified and written into a block, it can no longer be changed. On a second visit by the patient, the medical record performed at that time may be written in second block 1420. In some embodiment, the patients' medical records may be encoded so that the patients' medical records are only accessible to certain individuals with corresponding private key that decodes the encoded patient medical records. For instance, only the patient with a corresponding private key may access the medical record, thereby ensuring privacy.

In some embodiments, each medical record or block in a patient blockchain may additionally store information like the date, time, and dollar amount of the user's most recent fund transfer. Each medical record may additionally store information about who is participating in transactions, diagnosis, prescription, etc. Each medical record may additionally store information that distinguishes them from other blocks. Each medical record may store a unique code or hash that enable users to tell it apart from every other medical record. A single medical record on the blockchain may store up to 10 MB of data. Depending on the type of wallet 1310 and size of the transactions, a single medical record may house a few thousand transactions. For example, a crystal-based wallet, a desktop-based wallet, or hardware-based wallet may allow more transactions in a medical record to be stored because these types of wallets are executed on computers that contain larger memory.

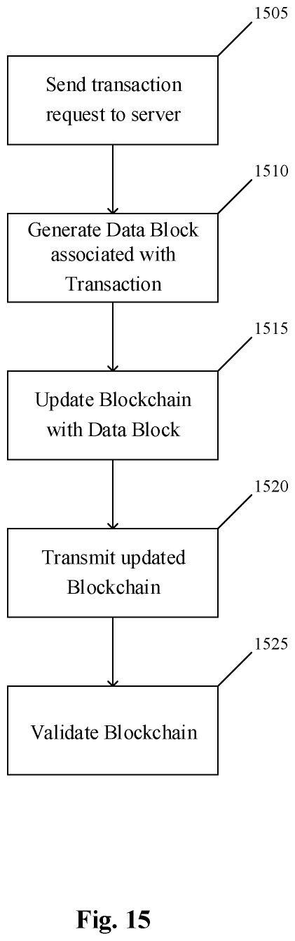

FIG. 15 illustrates a flowchart of an exemplary method for updating a blockchain, in accordance with an embodiment of the present invention. Method 1500 may begin with a step 1505 wherein a transaction may be sent to a server, for example, referring to FIG. 11, server 1112 or 1114, which is then distributed to all the nodes 1102 or 1104. The transaction may be any transaction depending on the blockchain to which it is being sent, such as, but not limited to, updating medical records, sending cryptocurrency, sending or receiving a transaction via radio wave or photonic beam, etc. The transaction may be sent to the server via a network connection 1124, for example. A data block associated with the transaction may be generated in a step 1510 of method 1500. For instance, before a medical record is generated, it may be verified by each node. If a majority of the nodes approve the transaction, then the transaction may be recorded into the data block. The data block may include various types of data, such as, but not limited to, transaction data, hash, user data, etc. Method 1500 may continue with a step 1515 in which a blockchain may be updated with the data block. To update the blockchain, proper hashing algorithms may be performed on the data block, and a hash of a last block in the blockchain may be included in the data block. In a step 1520 of method 1500, the updated blockchain may be transmitted via network connection to a distributed network of blockchain users or cryptocurrency wallets 1310. The updated blockchain may then be validated at a step 1525 of method 1500. The blockchain may be validated by any means known in the art, such as, but not limited to, proof-of-work, proof-of-stake, etc. When the transaction is validated, the transaction may be written to the blockchain. Otherwise, the transaction may be rejected and does not get written into the blockchain. Transactions that have been included in the blockchain may be considered as valid and final and cannot be changed. In some embodiment,

It will be further apparent to those skilled in the art that at least a portion of the novel method steps and/or system components of the present invention may be practiced and/or located in location(s) possibly outside the jurisdiction of the United States of America (USA), whereby it will be accordingly readily recognized that at least a subset of the novel method steps and/or system components in the foregoing embodiments must be practiced within the jurisdiction of the USA for the benefit of an entity therein or to achieve an object of the present invention. Thus, some alternate embodiments of the present invention may be configured to comprise a smaller subset of the foregoing means for and/or steps described that the applications designer will selectively decide, depending upon the practical considerations of the particular implementation, to carry out and/or locate within the jurisdiction of the USA.

For example, any of the foregoing described method steps and/or system components which may be performed remotely over a network (e.g., without limitation, a remotely located server) may be performed and/or located outside of the jurisdiction of the USA while the remaining method steps and/or system components (e.g., without limitation, a locally located client/node) of the forgoing embodiments are typically required to be located/performed in the USA for practical considerations. In client-server architectures, a remotely located server typically generates and transmits required information to a US based client/node, for use according to the teachings of the present invention. Depending upon the needs of the particular application, it will be readily apparent to those skilled in the art, in light of the teachings of the present invention, which aspects of the present invention can or should be located locally and which can or should be located remotely. Thus, for any claims construction of the following claim limitations that are construed under 35 USC § 112 (6)/(f) it is intended that the corresponding means for and/or steps for carrying out the claimed function are the ones that are locally implemented within the jurisdiction of the USA, while the remaining aspect(s) performed or located remotely outside the USA are not intended to be construed under 35 USC § 112 (6) pre-AIA or 35 USC § 112 (f) post AIA. In some embodiments, the methods and/or system components which may be located and/or performed remotely include, without limitation: any one or more of the operations described in connections of the systems and/or processes of the “A-Medicare” system and/or digital platform.

It is noted that according to USA law, all claims must be set forth as a coherent, cooperating set of limitations that work in functional combination to achieve a useful result as a whole. Accordingly, for any claim having functional limitations interpreted under 35 USC § 112 (6)/(f) where the embodiment in question is implemented as a client-server system with a remote server located outside of the USA, each such recited function is intended to mean the function of combining, in a logical manner, the information of that claim limitation with at least one other limitation of the claim.

For example, in client-server systems where certain information claimed under 35 USC § 112 (6)/(f) is/(are) dependent on one or more remote servers located outside the USA, it is intended that each such recited function under 35 USC § 112 (6)/(f) is to be interpreted as the function of the local system receiving the remotely generated information required by a locally implemented claim limitation, wherein the structures and or steps which enable, and breathe life into the expression of such functions claimed under 35 USC § 112 (6)/(f) are the corresponding steps and/or means located within the jurisdiction of the USA that receive and deliver that information to the client/node (e.g., without limitation, client-side processing and transmission networks in the USA). When this application is prosecuted or patented under a jurisdiction other than the USA, then “USA” in the foregoing should be replaced with the pertinent country or countries or legal organization(s) having enforceable patent infringement jurisdiction over the present patent application, and “35 USC § 112 (6)/(f)” should be replaced with the closest corresponding statute in the patent laws of such pertinent country or countries or legal organization(s).

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC § 112 (1), all claims must be supported by sufficient disclosure in the present patent specification, and any material known to those skilled in the art need not be explicitly disclosed. However, 35 USC § 112 (6) requires that structures corresponding to functional limitations interpreted under 35 USC § 112 (6) must be explicitly disclosed in the patent specification. Moreover, the USPTO's Examination policy of initially treating and searching prior art under the broadest interpretation of a “mean for” or “steps for” claim limitation implies that the broadest initial search on 35 USC § 112(6) (post AIA 112(f)) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC § 112(6) (post AIA 112(f)) when such corresponding structures are not explicitly disclosed in the foregoing patent specification.

Therefore, for any invention element(s)/structure(s) corresponding to functional claim limitation(s), in the below claims interpreted under 35 USC § 112(6) (post AIA 112(f)), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed.

Applicant(s) request(s) that fact finders during any claims construction proceedings and/or examination of patent allowability properly identify and incorporate only the portions of each of these documents discovered during the broadest interpretation search of 35 USC § 112(6) (post AIA 112(f)) limitation, which exist in at least one of the patent and/or non-patent documents found during the course of normal USPTO searching and or supplied to the USPTO during prosecution.

Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3^{rd} parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC § 112(6) (post AIA 112(f)), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC § 112 (6). Applicant(s) note that all the identified documents above which are incorporated by reference to satisfy 35 USC § 112 (6) necessarily have a filing and/or publication date prior to that of the instant application, and thus are valid prior documents to incorporated by reference in the instant application.

Having fully described at least one embodiment of the present invention, other equivalent or alternative methods of implementing a comprehensive digitally-based healthcare services procurement and delivery solution according to the present invention will be apparent to those skilled in the art. Various aspects of the invention have been described above by way of illustration, and the specific embodiments disclosed are not intended to limit the invention to the particular forms disclosed. The particular implementation of the comprehensive digitally-based healthcare services procurement and delivery solution may vary depending upon the particular context or application. By way of example, and not limitation, the comprehensive digitally-based healthcare services procurement and delivery solution described in the foregoing were principally directed to consumer healthcare related implementations; however, similar techniques may instead be applied to businesses and/or other private and/or non-private entities, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. That is, the Abstract is provided merely to introduce certain concepts and not to identify any key or essential features of the claimed subject matter. It is submitted with the understanding that it will not be used to limit or interpret the scope or meaning of the claims.

The following claims are hereby incorporated into the detailed description, with each claim standing on its own as a separate embodiment.

Only those claims which employ the words “means for” or “steps for” are to be interpreted under 35 USC 112, sixth paragraph (pre-AIA) or 35 USC 112(f) post-AIA. Otherwise, no limitations from the specification are to be read into any claims, unless those limitations are expressly included in the claims.

What is claimed is:

1. A non-transitory computer-readable storage medium with a computer program code stored thereon, wherein the computer program code instructs a processor to perform a method comprising:
loading an application software stored on a node, said application software is configured to execute the following steps;
connecting to a network;
registering as a member node of the network;
determining a hash of a public key broadcasted on the network;
based on said determining step, matching said public key with an address of a wallet stored on said member node, said wallet comprises at least one of a mobile phone-based wallet, a desktop-based wallet, a hardware-based wallets, a crystal-based wallets;
based on said matching step, decrypting said public key;
comparing said decrypted public key to a private key stored in said wallet; and
based on said comparing step, sending a predetermined amount of cryptocurrency to a sender of said public key.

2. The method of claim 1, further comprising the steps of broadcasting a transaction performed in said sending step.

3. The method of claim 2, further comprising the steps of receiving a verification of the transaction, wherein the transaction is verified by other member nodes of the network.

4. The method of claim 3, based on a result of said verification step, the transaction performed in said sending step is recorded into a data record.

5. The method of claim 4, wherein said data record comprises a blockchain that is configured to maintain a continuously growing list of transactions written into blocks, said blockchain comprises at least one or more block that are interconnected together, wherein each block contains a hash to cryptographically secure said block from tampering and revision.

6. The method of claim 5, in which said recording step comprises writing said transaction into a new block that comprises said blockchain and a hash of the new block, wherein succeeding transactions are written into connecting blocks in the order in which they are received.

7. The method of claim 6, wherein said succeeding blocks comprises a hash of the current block and a hash of the previous block.

8. The method of claim 7, wherein said each block further comprises an address.

9. The method of claim 2, in which said transaction comprises a medical record of a patient, wherein said medical record comprises at least a patient name, date of birth, a diagnosis, and date of diagnosis.

10. The method of claim 9, further comprising the steps of broadcasting the medical record of the patient to other member nodes for certification.

11. The method of claim 10, further comprising the steps of receiving said certification of the patients' medical record, wherein the patients' medical record is verified by the other member nodes of the network.

12. The method of claim 11, based on a result of said certification step, the patients' medical record is written into a data record of said wallet.

13. The method of claim 12, wherein said data record comprises a blockchain that is configured to maintain a continuously growing list of medical records written into blocks.

14. The method of claim 13, in which said recording step comprises writing said medical record into a new block that comprises said blockchain, wherein said new block further comprises a recording timestamp and a hash of said new block.

15. The method of claim 14, wherein succeeding medical records are written into blocks in the order in which they are received, in which each succeeding medical record comprises a hash of the current medical record and a hash of the previous medical record, and in which each succeeding medical record further comprises a recording timestamp.

16. The method of claim 15, wherein each medical record is encoded, and wherein said encoding is configured to make each medical record accessible to member nodes with corresponding private key.

17. A non-transitory computer-readable storage medium with a computer program code stored thereon, wherein the computer program code instructs a processor to perform a method comprising the steps of:
connecting to a network;
registering as a member node of the network;
determining a hash of a public key broadcasted on the network;
based on said determining step, matching said public key with an address of a wallet stored on said member node, said wallet comprises at least one of a mobile phone-based wallet, a desktop-based wallet, a hardware-based wallets, a crystal-based wallets;
based on said matching step, decrypting said public key;
comparing said decrypted public key to a private key stored in said wallet;
based on said comparing step, sending a predetermined amount of cryptocurrency to a sender of said public key;
broadcasting a transaction performed in said sending step for verification, wherein the transaction is verified by other member nodes of the network; and
based on a result of said verification step, the transaction performed in said sending step is recorded into a data record, wherein said data record comprises a blockchain that is configured to maintain a continuously growing list of transactions written into blocks, and wherein said blockchain comprises at least one or more block that are interconnected together.

18. The method of claim 17, in which said recording step comprises writing said transaction into a block that comprises said blockchain, wherein succeeding transactions are written into connecting blocks in the order in which they are received, and wherein each block contains a hash to cryptographically secure each block from tampering and revision.

19. A non-transitory computer-readable storage medium with a computer program code stored thereon, wherein the computer program code instructs a processor to perform a method comprising:
loading an application software stored on a node, said application software is configured to execute the following steps;
connecting to a network;
registering as a member node of the network;
determining a hash of a public key broadcasted on the network;
based on said determining step, matching said public key with an address of a wallet stored on said member node, said wallet comprises at least one of a mobile phone-based wallet, a desktop-based wallet, a hardware-based wallets, a crystal-based wallets;
based on said matching step, decrypting said public key;
comparing said decrypted public key to a private key stored in said wallet;
based on said comparing step, sending a predetermined amount of cryptocurrency to a sender of said public key;
broadcasting a medical record to other member nodes of the network for certification, wherein said medical record comprises at least a patient name, date of birth, a diagnosis, and date of diagnosis;
receiving a result of said certification of the medical record, wherein the patients' medical record is verified by the other member nodes of the network;
based on said certification, the patients' medical record is written into a data record of said wallet, wherein said data record comprises a blockchain; and
in which said writing step comprises writing said medical record into a block that comprises said blockchain, wherein said new block further comprises a recording timestamp and a hash of said new block.

20. The method of claim 19, wherein succeeding medical records are written into blocks in the order in which they are received, in which each succeeding medical record comprises a hash of the current medical record and a hash of the previous medical record, and in which each succeeding medical record further comprises a recording timestamp, wherein each medical record is encoded, and wherein said encoding is configured to make each medical record accessible to member nodes with corresponding private key.
